(19) United States
(12) Patent Application Publication
(10) Pub. No.: US 2010/0098641 A1
(43) Pub. Date: Apr. 22, 2010

(54) Monophosphates as Mutual Prodrugs of Anti-Inflammatory Signal Transduction Modulators (AISTM's) and Beta-Agonists for the Treatment of Pulmonary Inflammation and Bronchoconstriction

(76) Inventors: William Baker, Bellevue, WA (US); Marcin Stasiak, Seattle, WA (US); Sundaramoorthi Swaminathan, Burlingame, CA (US); Musong Kim, Bothell, WA (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/519,305

(22) PCT Filed: Dec. 12, 2007
(86) PCT No.: PCT/US2007/025361

§ 371 Date: Nov. 4, 2009

Related U.S. Application Data
(60) Provisional application No. 60/874,543, filed on Dec. 13, 2006.

(51) Int. Cl. A61K 9/12; C07F 9/06

(52) U.S. Cl. 424/45; 546/23; 544/243; 544/119; 568/15

(57) ABSTRACT

A mutual prodrug of an AISTM and a β-agonist in formulation for delivery by aerosolization to inhibit pulmonary inflammation and bronchoconstriction is described. The mutual prodrug is preferably formulated in a small volume solution (10-500 μL) dissolved in a quarter normal saline having pH between about 5.0 and 7.0 for the treatment of respiratory tract inflammation and bronchoconstriction by an aerosol having mass median average diameter predominantly between about 1 to 5μ, produced by nebulization or by dry powder inhaler.

### CROSS REFERENCE TO RELATED APPLICATION

This application claims the priority of U.S. Provisional Application No. 60/874,543, filed Dec. 13, 2006.

### FIELD OF THE INVENTION

The current invention relates to the preparation of novel, mutual prodrugs of anti-inflammatory signal transduction modulators (AISTM's) and β-agonists for delivery to the lung by aerosolization. In particular, the invention concerns the synthesis, formulation and delivery of monophosphates as mutual AISTM-β-agonist prodrugs such, that when delivered to the lung, endogenous enzymes present in the lung tissue and airways degrade the mutual prodrug releasing an AISTM and a β-agonist (e.g. salmeterol, albuterol) at the site of administration. The described mutual prodrugs are formulated as either liquids or dry powders and the formulation permits and is suitable for delivery of the prodrugs to the lung endobronchial space of airways in an aerosol having a mass median average diameter predominantly between 1 to 5μ. The formulated and delivered efficacious amount of monophosphate prodrugs is sufficient to deliver therapeutic amounts of both AISTM and β-agonist for treatment of respiratory tract diseases, specifically pulmonary inflammation and bronchoconstriction associated with mild to severe asthma, as well as chronic bronchitis or chronic obstructive pulmonary disease (COPD).

### BACKGROUND OF THE INVENTION

Asthma is a chronic inflammatory disease of the airways resulting from the infiltration of pro-inflammatory cells, mostly eosinophils and activated T-lymphocytes into the bronchial mucosa and submucosa. The secretion of potent chemical mediators, including cytokines, by these proinflammatory cells alters mucosal permeability, mucus production, and causes smooth muscle contraction. All of these factors lead to an increased reactivity of the airways to a wide variety of irritant stimuli (Kaliner, 1988).

Targeting signal transduction pathways is an attractive approach to treating inflammatory diseases, as the same pathways are usually involved in several cell types and regulate several coordinated inflammatory processes, hence modulators have the prospect of a wide spectrum of beneficial effects. Multiple inflammatory signals activate a variety of cell surface receptors that activate a limited number of signal transduction pathways, most of which involve cascades of kinases. These kinases in turn may activate transcription factors that regulate multiple inflammatory genes. Applying “anti-inflammatory signal transduction modulators” (referred to in this text as AISTM), like phosphodiesterase inhibitors (e.g. PDE-4, PDE-5, or PDE-7 specific), transcription factor inhibitors (e.g. blocking NFκB through IKK inhibition), or kinase inhibitors (e.g. blocking P38 MAP, JNK, PI3K, EGFR or Syk) is a logical approach to switching off inflammation as these small molecules target a limited number of common intracellular pathways—those signal transduction pathways that are critical points for the anti-inflammatory therapeutic intervention (see review by P. J. Barnes, 2006).

Unfortunately, this same advantage is also a disadvantage as the widespread distribution of the same signal transduction pathways means that modulators have a high risk of dose-limiting adverse side effects (e.g. nausea, diarrhea, headaches, immune deficiency and arteriopathy observed for PDE-4 inhibitors) due to lack of cell and effect specificity. A potential solution to systemic side effects would be the delivery of such AISTM drugs directly to the site of inflammation, i.e. via inhalation delivery to lungs in case of treatment of diseases related to pulmonary inflammation. However many existing AISTM's were developed targeting oral delivery, therefore they posses good absorption properties, which can likely lead to unwanted systemic exposure via absorption from lungs into circulation. The prodrug strategy however, could be a more effective solution, rendering high lung retention, poor systemic absorption and sustained-release properties that could be engineered into the chemical entity delivered directly into site of inflammation (i.e. lungs).

Bronchodilators such as albuterol or salmeterol relax airway smooth muscles by blocking active contraction. Many of these bronchodilators activate the β₂-adrenoreceptor as their mode of action. The result is the dilation by 2-3 mm in diameter of small peripheral airways, which are the site of action in both asthma and COPD.

In consideration of all problems and disadvantages connected with the adverse side effect profile of AISTM's (e.g. nausea, diarrhea, vasculitis, immune suppresion) and of β-agonists (e.g. tachycardia, ventricular dysrhythmias, hypokalemia) it would be highly advantageous to provide a water-soluble, mutual AISTM-β-agonist prodrug to mask the pharmacological properties of both AISTM and β-agonists until such a prodrug reaches lungs, thereby mitigating the systemic side effects of AISTM's and cardiovascular side-effects of β-agonists. Such a mutual AISTM-β-agonist prodrug would be effectively delivered to the endobronchial space and then converted to active drugs by the action of lung enzymes, thereby delivering to the site of inflammation and bronchoconstriction a therapeutic amount of both drugs.

The mutual AISTM-β-agonist prodrug would provide a therapeutic agent to dilate the airway, thereby allowing the second component (AISTM) to effectively penetrate and reach the site of inflammation. It would be highly desired to have a mutual prodrug of a β-agonist and an AISTM that produces sustained release of both drugs at the site of administration. Additionally, it would be highly desirable to have such a mutual prodrug to be poorly absorbed from the lung and to be sufficiently water soluble to allow flexibility in its formulation and delivery system.

It is therefore a primary object of this invention to provide novel monophospates as mutual prodrugs of an AISTM and a β-agonist.

It is a further object of this invention to provide a composition of such mutual prodrugs, which is stable as a liquid or solid dosage form for nebulization or dry powder delivery. Such composition contains sufficient but not excessive concentration of the active substance which can be efficiently aerosolized by metered-dose inhalers, nebulization in jet, ultrasonic, pressurized, or vibrating porous plate nebulizers or by dry powder into aerosol particles predominantly within the 1 to 5μ size range, wherein the salinity and pH are adjusted to permit generation of a mutual prodrug aerosol well tolerated by patients, and the formulation has an adequate shelf life.

### SUMMARY OF THE INVENTION

The present invention is directed to monophosphates as mutual prodrugs of AISTM's and β-agonist and their use and formulation for delivery by inhalation as a method to treat pulmonary inflammation and bronchoconstriction. The prodrug incorporates a polar (charged in physiologic pH) phosphate and a quaternary nitrogen atom (positively charged), which renders the molecule highly polar, enhances its hydrophilicity and imparts its affinity to lung DNA and protein thus minimizing rapid systemic absorption, as well as absorption due to swallowing. Furthermore, since the mutual prodrug cannot be activated in the absence of alkaline phosphatase, the systemic side effects are eliminated due to the minimal activity of that enzyme in saliva (in the case of partial mutual prodrug deposition in mouth) and due to low phosphatase activity in plasma, as compared to other tissues, particularly lungs (Testa and Mayer, 2003).

More specifically, the present invention is directed to a compound of the formula A

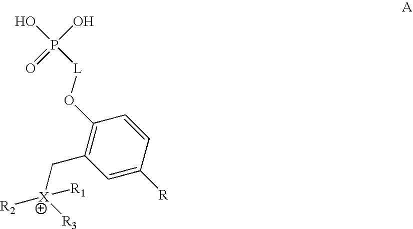

and pharmaceutical acceptable salts thereof, wherein:
X represents a quaternizable moiety, i.e. nitrogen or sulfur atom or a nitrogen-containing heterocycle;
R₁R₂R₃X taken together represents an anti-inflammatory signal transduction modulator (AISTM—i.e. a phosphodiesterase inhibitor, a kinase inhibitor, transcription factor inhibitor) or its prodrug (e.g. ester) linking the parent molecule possessing AISTM activity to a quaternizable moiety X;
L is a bond or methyleneoxy- (CH₂O) group;

### R is

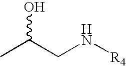

where R₄ is an alkyl group of 1-12 carbon atoms, arylalkyl or substituted arylalkyl where 1-3 CH₂ groups in the carbon chain may be replaced with atom(s) selected from O, S and NR₅ where R₅ is hydrogen or alkyl.

In a preferred embodiment, the prodrug linking the parent molecule possessing AISTM activity to a quaternizable moiety X is an acetyl ester, In another preferred embodiment, the prodrug linking the parent molecule possessing AISTM activity to a quaternizable moiety X is an acetyloxymethyl ester,

Presently preferred embodiments of this invention include compounds of formula A, wherein:

### R is

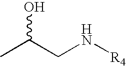

where R₄ is (CH₂)₆O(CH₂)₄Ph or tert-butyl,
L is a bond,
and R₁R₂R₃X taken together represent an anti-inflammatory signal transduction modulator (AISTM) such as:
5-(2,4-Difluoro-phenoxy)-1-isobutyl-1H-indazole-6-carboxylic acid (2-dimethylamino-ethyl)-amide (P38 Map kinase inhibitor ARRY-797);
3-Cyclopropylmethoxy-N-(3,5-dichloro-pyridin-4-yl)-4-difluorormethoxy-benzamide (PDE-4 inhibitor Roflumilast);
4-[2-(3-cyclopentyloxy-4-methoxyphenyl)-2-phenyl-ethyl]-pyridine (PDE-4 inhibitor CDP-840);
N-(3,5-dichloro-4-pyridinyl)-4-(difluoromethoxy)-8-[(methylsulfonyl)amino]-1-dibenzofurancarboxamide (PDE-4 inhibitor Oglemilast);
N-(3,5-Dichloro-pyridin-4-yl)-2-[1-(4-fluorobenzyl)-5-hydroxy-1H-indol-3-yl]-2-oxo-acetamide (PDE-4 inhibitor AWD 12-281);
8-Methoxy-2-trifluoromethyl-quinoline-5-carboxylic acid (3,5-dichloro-1-oxy-pyridin-4-yl)-amide (PDE-4 inhibitor Sch 351591);
4-[5-(4-Fluorophenyl)-2-(4-methanesulfinyl-phenyl)-1H-imidazol-4-yl]-pyridine (P38 inhibitor SB-203850);
4-[4-(4-Fluoro-phenyl)-1-(3-phenyl-propyl)-5-pyridin-4-yl-1H-imidazol-2-yl]-but-3-yn-1-ol (P38 inhibitor RWJ-67657);
4-Cyano-4-(3-cyclopentyloxy-4-methoxy-phenyl)-cyclohexanecarboxylic acid 2-diethylamino-ethyl ester (2-diethyl-ethyl ester prodrug of Cilomilast, PDE-4 inhibitor);
(3-Chloro-4-fluorophenyl)-[7-methoxy-6-(3-morpholin-4-yl-propoxy)-quinazolin-4-yl]-amine (Gefitinib, EGFR inhibitor); and
4-(4-Methyl-piperazin-1-ylmethyl)-N-[4-methyl-3-(4-pyridin-3-yl-pyrimidin-2-ylamino)-phenyl]-benzamide (Imatinib, EGFR inhibitor).

Examples of presently preferred compounds of this invention include:
(2-{[5-(2,4-Difluoro-phenoxy)-1-isobutyl-1H-indazole-6-carbonyl]-amino}-ethyl)-(5-{1-hydroxy-2-[6-(4-phenyl-butoxy)-hexylamino]-ethyl}-2-phosphonooxy-benzyl)-dimethyl-ammonium (Example 29);
[5-(2-tert-Butylamino-1-hydroxy-ethyl)-2-phosphonooxy-benzyl]-(2-{[5-(2,4-difluoro-phenoxy)-1-isobutyl-1H-indazole-6-carbonyl]-amino}-ethyl)-dimethyl-ammonium (Example 30);
4-[2-(3-Cyclopentyloxy-4-methoxy-phenyl)-2-phenyl-ethyl]-1-(4-{1-hydroxy-2-[6-(4-phenyl-butoxy)-hexylamino]-ethyl}-2-phosphonooxy-benzyl)-pyridinium (Example 37);
[4-(2-tert-Butylamino-1-hydroxy-ethyl)-2-phosphonooxy-benzyl]-4-[2-(3-cyclopentyloxy-4-methoxy-phenyl)-2-phenyl-ethyl]-pyridinium (Example 38);
3,5-Dichloro-4-[(4-difluoromethoxy-8-methanesulfonylamino-dibenzofuran-1-carbonyl)-amino]-1-(4-{1-hydroxy-2-[6-(4-phenyl-butoxy)-hexylamino]-ethyl}-2-phosphonooxy-benzyl)-pyridinium (Example 57); and
1-[4-(2-tert-Butylamino-1-hydroxy-ethyl)-2-phosphonooxy-benzyl]-3,5-dichloro-4-[(4-difluoromethoxy-8-methanesulfonylamino-dibenzofuran-1-carbonyl)-amino]-pyridinium (Example 58).

The present invention also relates to processes of synthesis of the preferred mutual prodrugs listed above.

The invention also relates to a pharmaceutically acceptable composition for the treatment of a disorder selected from severe to mild asthma, chronic bronchitis, COPD or other diseases related to pulmonary inflammation and bronchoconstriction, which comprises a therapeutically effective amount, preferably from about 10 μg to about 1000 μg, of at least one compound of formula A or a pharmaceutically acceptable salt thereof, and a pharmaceutically acceptable carrier. The composition is preferably administered as an aerosol, most preferably by a dry powder inhaler. The invention also relates to methods of treating such diseases with therapeutically effective amounts of at least one compound of formula A or a pharmaceutically acceptable salt thereof.

The invention also relates to a liquid or dry powder formulation of a compound of Formula A for the treatment of a disorder selected from severe to mild asthma, chronic bronchitis and COPD or other diseases related to pulmonary inflammation and bronchoconstriction, which comprises a therapeutically effective amount, preferably from about 10 μg to about 1000 μg, of at least one compound of formula A or a pharmaceutically acceptable salt thereof. The composition is preferably administered as an aerosol, most preferably by a dry powder inhaler.

The invention further relates to a method for the prevention and treatment of pulmonary inflammation and bronchoconstriction, comprising administering to a patient in need of such treatment an effective amount of an aerosol formulation comprising about 10 μg to about 1000 μg of at least one compound of Formula A. Preferably, when the compound of Formula A is delivered to the lung, the phosphate group is cleaved by an endogenous enzyme alkaline phosphatase and the AISTM and the β-agonist are individually released in a simultaneous manner.

### DETAILED DESCRIPTION OF THE INVENTION

As used herein “aryl” is defined as a C₆-C₁₈ carbocyclic ring that may be substituted with 1-3 groups selected from hydrogen, amino, hydroxy, halo, O-alkyl and NH-alkyl. Aryl can be one or two rings either fused to form a bicyclic aromatic ring system or linear as in biphenyl. One or more of the carbon atoms in an aryl group can optionally be replaced by N, S, or O in the ring to produce a heterocyclic system.

The term “alkyl” as used herein refers to a branched or straight chain comprising one to twenty carbon atoms, at least one of which can optionally be replaced by an atom selected from O, S, or NR₅ where R₅ is as defined herein. Representative alkyl groups include methyl, butyl, hexyl, and the like.

As used herein “lower alkyl” includes both substituted or unsubstituted straight or branched chain alkyl groups having from 1 to 10 carbon atoms. Representative lower alkyl groups include for example, methyl, ethyl, propyl, isopropyl, n-butyl, tert-butyl, and the like. Representative halo-substituted, amino-substituted and hydroxy-substituted, lower-alkyl groups include chloromethyl, chloroethyl, hydroxyethyl, aminoethyl, etc.

As used herein “cycloalkyl” includes a non-aromatic ring composed of 3-10 carbon atoms.

As used herein, the term “halogen” refers to chloro, bromo, fluoro and iodo groups.

The term “substituted heterocycle” or “heterocyclic group” or “heterocycle” as used herein refers to any 3- or 4-membered ring containing a heteroatom selected from nitrogen, oxygen, and sulfur or a 5- or 6-membered ring containing from one to three heteroatoms selected from the group consisting of nitrogen, oxygen, or sulfur; wherein the 5-membered ring has 0-2 double bounds and the 6-membered ring has 0-3 double bounds; wherein the nitrogen and sulfur atom may be optionally oxidized; wherein the nitrogen and sulfur heteroatoms may be optionally quarternized; and including any bicyclic group in which any of the above heterocyclic rings is fused to a benzene ring or another 5- or 6-membered heterocyclic ring independently as defined above. Heterocyclics in which nitrogen is the heteroatom are preferred. Fully saturated heterocyclics are also preferred. Preferred heterocycles include: diazapinyl, pyrryl, pyrrolinyl, pyrrolidinyl, pyrazolyl, pyrazolinyl, pyrazolidinyl, imidazoyl, imidazolinyl, imidazolidinyl, pyridyl, piperidinyl, pyrazinyl, piperazinyl, azetidinyl, pyrimidinyl, pyridazinyl, oxazolyl, oxazolidinyl, isoxazolyl, isoazolidinyl, morpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, triazolyl and benzothienyl groups.

Heterocyclics can be unsubstituted or monosubstituted or disubstituted with substituents independently selected from hydroxy, halo, oxo (C═O), alkylimino (RN═, wherein R is a lower alkyl or alkoxy group), amino, alkylamino, dialkylamino, acylaminoalkyl, alkoxy, thioalkoxy, loweralkyl, cycloalkyl or haloalkyl. The most preferred heterocyclics include imidazolyl, pyridyl, piperazinyl, azetidinyl, thiazolyl, triazolyl, benzimidazolyl, benzothiazolyl and benzoxazolyl.

As used herein, the term “pharmaceutically acceptable salts” refers to the salt with a nontoxic acid or alkaline earth metal salt of the compounds of formula A. These salts can be prepared in situ during the final isolation and purification of the compounds of formula A, or separately, by reacting the base or acid functions with a suitable organic or inorganic acid or base, respectively. Representative acid salts include hydrochloride, hydrobromide, bisulfate, acetate, oxalate, valerate, oleate, palmitate, stearate, laurate, borate, benzoate, lactate, citrate, maleate, tartrate salts, and the like. Representative alkali metals of alkaline earth metal salts include sodium, potassium, calcium, and magnesium.

As used herein, the term “alkoxy” refers to —O—R wherein R is lower alkyl as defined above. Representative examples of lower alkoxy groups include methoxy, ethoxy, tert-butoxy, and the like.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

The term “normal saline” means water solution containing 0.9% (w/v) NaCl.

The term “diluted saline” means normal saline containing 0.9% (w/v) NaCl diluted into its lesser strength.

The term “quarter normal saline” or “¼NS” means normal saline diluted to its quarter strength containing 0.225% (w/v) NaCl.

The term “prodrug” as used herein refers to a compound in which specific bond(s) of the compound are broken or cleaved by the action of an enzyme or by biological process thereby producing or releasing a drug and compound fragment which is substantially biologically inactive. A prodrug is thus a covalently modified analog or latent form of a therapeutically active compound.

Typical examples of prodrugs of the compounds of the invention have biologically labile protecting groups on a functional moiety of the compound. Prodrugs include compounds that can be oxidized, reduced, aminated, deaminated, esterified, deesterified, alkylated, dealkylated, acylated, deacylated, phosphorylated, dephosphorylated, photolyzed, hydrolyzed, or other functional group change or conversion involving forming or breaking chemical bonds on the prodrug.

“Prodrug moiety” means a labile functional group which separates from the active inhibitory compound during metabolism, systemically, inside a cell, by hydrolysis, enzymatic cleavage, or by some other process (Bundgaard, Hans, “Design and Application of Prodrugs” in Textbook of Drug Design and Development (1991), P. Krogsgaard-Larsen and H. Bundgaard, Eds. Harwood Academic Publishers, pp. 113-191). Enzymes which are capable of an enzymatic activation mechanism with the prodrug compounds of the invention include, but are not limited to, amidases, esterases, microbial enzymes, phospholipases, cholinesterases, and phosphases. Prodrug moieties can serve to enhance solubility, absorption and lipophilicity to optimize drug delivery, bioavailability and efficacy.

Exemplary prodrug moieties include the hydrolytically sensitive or labile acyl esters —OC(═O)R⁹, acyloxymethyl esters —CH₂C(═O)R⁹ and acyloxymethyl carbonates —CH₂OC(═O)OR⁹ where R⁹ is C₁-C₆ alkyl, C₁-C₆ substituted alkyl, C₆-C₂₀ aryl or C₆-C₂₀ substituted aryl. In some instances, the R⁹ group will contain a hydrolytically sensitive group such as a quanternary amine which is also hydrolytically labile. The acyloxyalkyl ester was first used as a prodrug strategy for carboxylic acids and then applied to phosphates and phosphonates by Farquhar et al (1983) J. Pharm. Sci. 72: 324; also U.S. Pat. Nos. 4,816,570, 4,968,788, 5,663,159 and 5,792,756. A close variant of the acyloxyalkyl ester, the alkoxycarbonyloxyalkyl ester (carbonate), may also act as a prodrug moiety in the compounds of this invention. An exemplary acyloxymethyl ester is pivaloyloxymethoxy, (POM) —CH₂C(═O)C(CH₃)₃. An exemplary acyloxymethyl carbonate prodrug moiety is pivaloyloxymethylcarbonate (POC) —CH₂C(═O)OC(CH₃)₃.

The term “mutual prodrug” as used herein refers to a bipartite or tripartite prodrug in which specific bond(s) of the compound are broken or cleaved by the action of an enzyme or by biological process thereby producing or releasing two or more drugs or prodrugs.

Unless otherwise stated, it is understood that, whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

The compounds of the invention may comprise asymmetrically substituted carbon atoms. Such asymmetrically substituted carbon atoms can result in the compounds of the invention comprising mixtures of stereoisomers at a particular asymmetrically substituted carbon atom or a single stereoisomer. As a result, racemic mixtures, mixtures of diastereomers, as well as single diastereomers of the compounds of the invention are included in the present invention. The terms “S” and “R” configuration, as used herein, are as defined by the IUPAC 1974 RECOMMENDATIONS FOR SECTION E, FUNDAMENTAL STEREOCHEMISTRY, Pure Appl. Chem. 45:13-30 (1976). The terms α and β are employed for ring positions of cyclic compounds. The α-side of the reference plane is that side on which the preferred substituent lies at the lower numbered position. Those substituents lying on the opposite side of the reference plane are assigned β descriptor. It should be noted that this usage differs from that for cyclic stereoparents, in which “a” means “below the plane” and denotes absolute configuration. The terms α and β configuration, as used herein, are as defined by the CHEMICAL ABSTRACTS INDEX GUIDE-APPENDIX IV (1987) paragraph 203.

The present invention also relates to processes for preparing the compounds of the invention and to the synthetic intermediates useful in such processes, as described in detail below.

### I. Preparation of the Compounds of the Invention

The compounds of the present invention can be prepared by the processes illustrated in Schemes I-VI.

A convergent route to compounds of Formula A involves:

a) synthesis of the phosphorylated β-agonist derivatives activated towards alkylation (Scheme I-V); and
b) quaternization (alkylation) of the AISTM molecule or their physiologically cleavable esters carrying a “quaternizable moiety”, with the activated P-agonist derivative, followed by the final deprotection (Scheme VI).

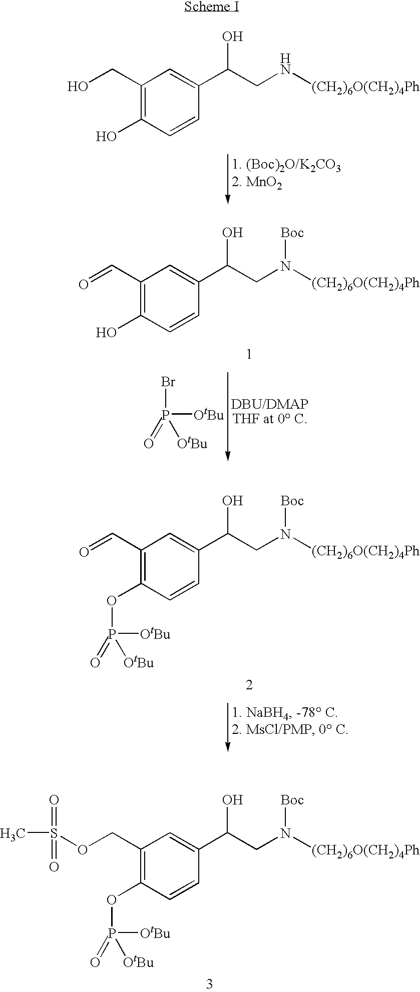

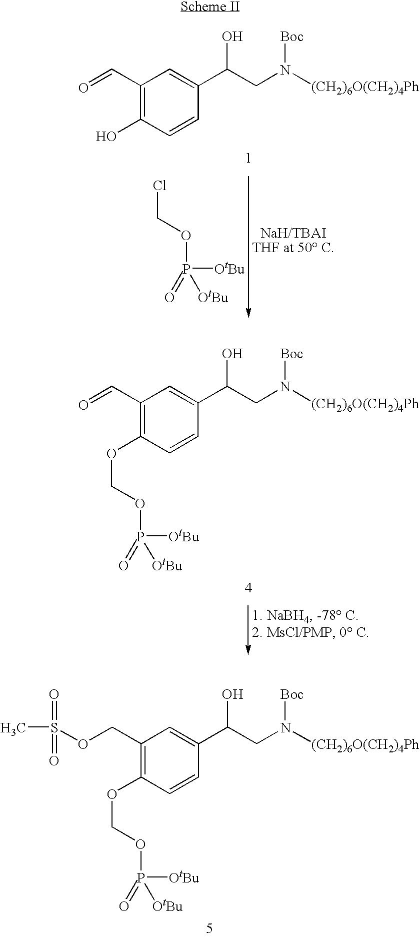

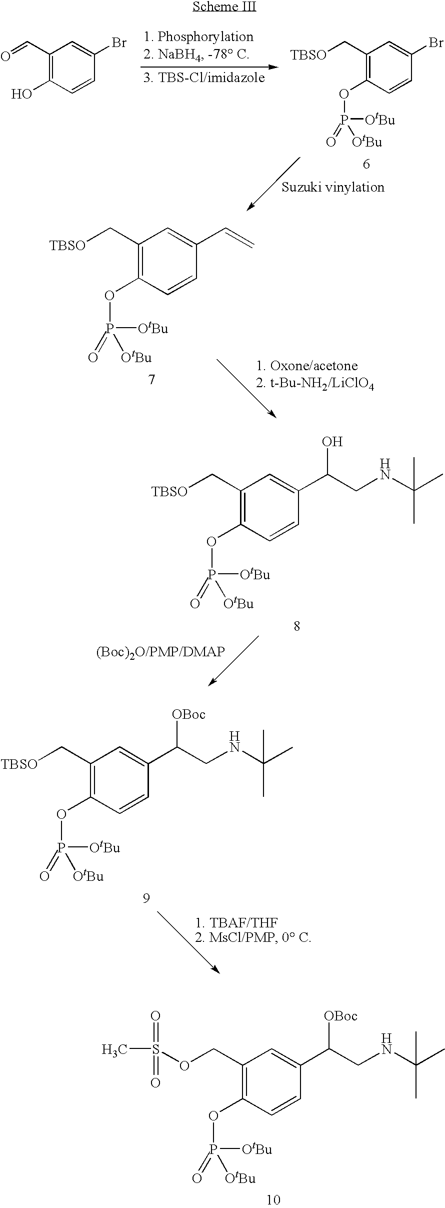

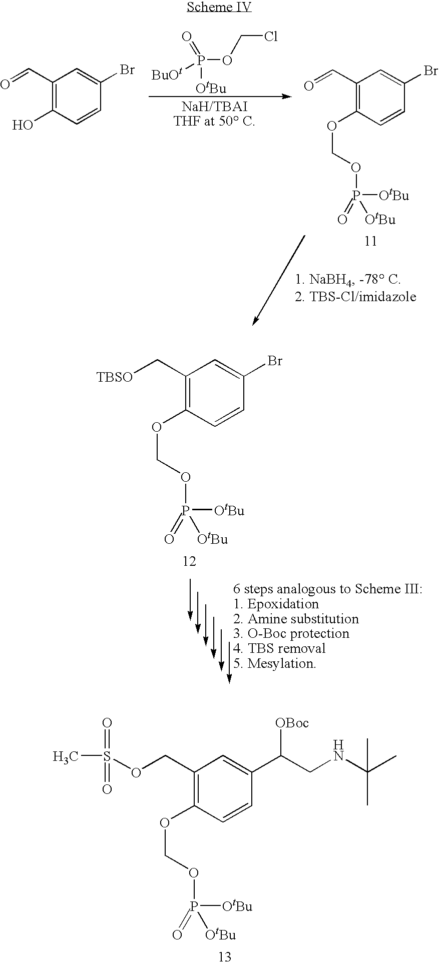

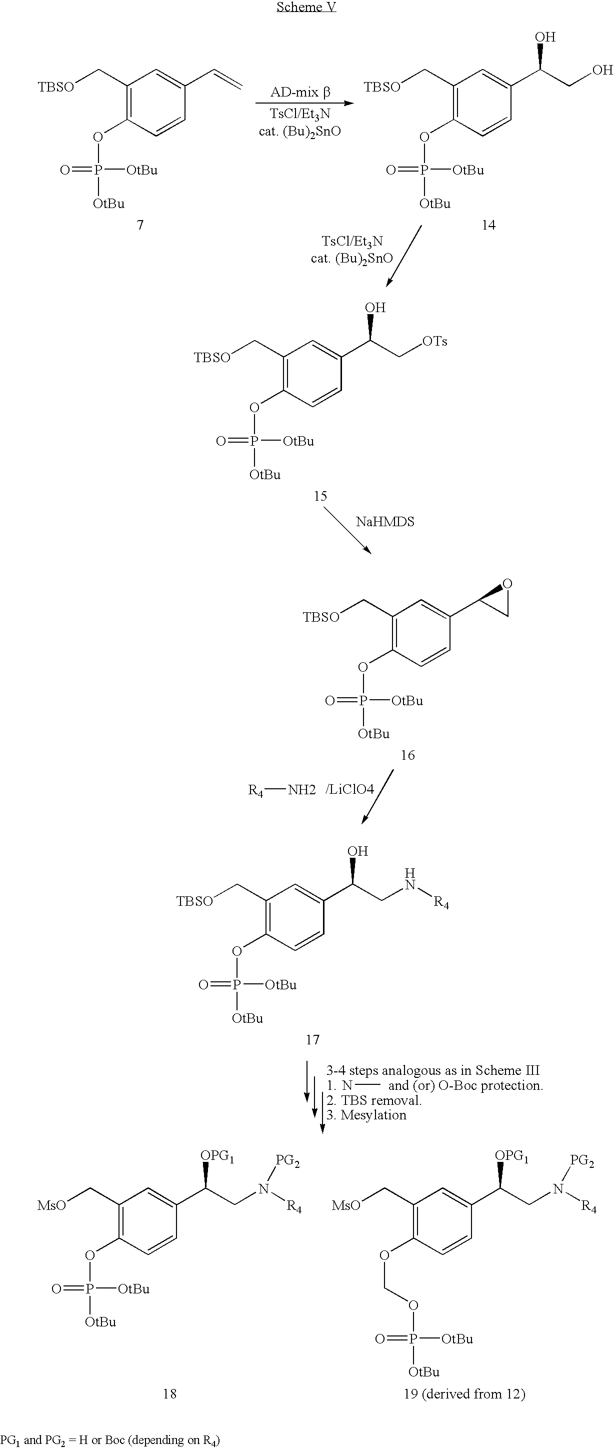

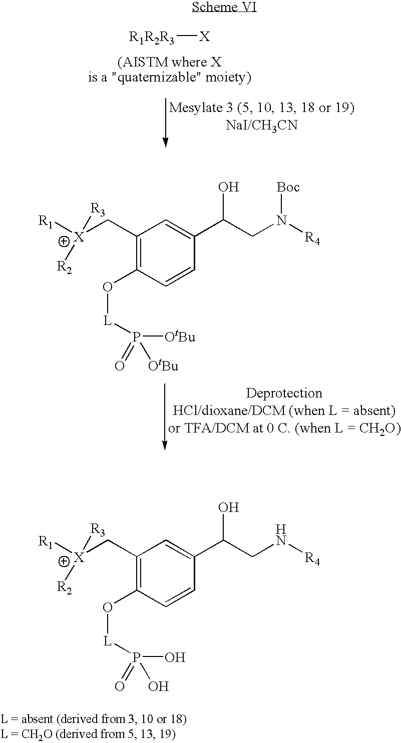

The synthesis of the phosphate-functionalized protected β-agonist derivatives is shown in Schemes I-V.

Commercially available racemic salmeterol xinafoate (or prepared according to Rong and Ruoho, 1999) is protected with a t-butoxycarbonyl group (Boc), followed by the selective oxidation of the primary, benzylic alcohol to aldehyde with activated MnO₂, yielding compound 1 (Example 3). In this manner the primary alcohol is disguised as an aldehyde and therefore the acidity of the phenolic moiety is increased, helping the selectivity of the subsequent phosphorylation. As a consequence the reaction with a slight excess of phosphobromidate (prepared as described in Example 1) proceeds cleanly, yielding the phosphate 2 in good yield and purity (Example 4). The reduction of the aldehyde moiety with sodium borohydride carried out at low temperature (−78° C. to 0° C.) produces the diol, which is selectively sulfonylated at 0° C. using methanesulfonyl chloride (MsCl) in the presence of 1,2,2,6,6-pentamethylpiperidine (PMP) to give the primary mesylate 3 (Example 6). Thus activated intermediate (Scheme I) is used in the alkylations linking the AISTM molecule and a β-agonist into a mutual prodrug as depicted in Scheme VI.

Alternatively, the phosphono-oxymethyl derivative of salmeterol can be prepared as described in Scheme II. The phenolic moiety in compound 1 is alkylated at 50° C. with di-tert-butyl chloromethyl phosphate (Krise et al., 1999) using sodium hydride as a base and tetrabutylammonium iodide as an auxiliary, yielding the derivative 4. The borohydride reduction of aldehyde, followed by the selective mesylation of the primary hydroxyl group (analogously as described in the preceding paragraph) gives the activated mesylate 5.

In the preparation of albuterol derivative, the steric bulk around the aminoalcohol moiety (R₄=t-butyl) requires the indirect synthetic approach illustrated in Scheme III.

5-Bromosalicylaldehyde is phosphorylated and the aldehyde moiety reduced as described in the earlier paragraph, and the thus formed alcohol moiety is protected by treatment with tert-butyldimethylsilyl chloride in the presence of imidazole, yielding compound 6 (Examples 10-11). The presence of bromine atom allows the C—C bond formation in the following step. The trivinylboroxine-pyridine complex in the presence of catalytic amounts of tricyclohexylphosphine and palladium (II) acetate is used to introduce the vinyl substituent using the Suzki method (Example 12). Thus formed compound 7 undergoes the epoxidation by means of 2,2-dimethyldioxirane (DMDO) generated in situ in a mixture of oxone and acetone. The epoxide opening is accomplished by nucleophilic attack with tert-butylamine in the presence of lithium perchlorate as a Lewis acid ensuring regioselectivity resulting with beta-aminoalcohol 8. Steric bulk imposed by the t-butyl moiety has impact on the subsequent acylation with di-t-butyl dicarbonate, which proceeds selectively on the secondary hydroxyl, rather than the secondary amine, yielding compound 9. The removal of silyl TBS protection is followed by low-temperature mesylation, which again, proceeds selectively on the primary, benzylic hydroxyl, producing mesylate 10 (with the hindered, secondary t-butylamine moiety untouched).

Alternatively, the phosphono-oxymethyl derivative of albuterol can be prepared as described in Scheme IV. The phenolic moiety in 5-bromosalicaldehyde is alkylated at 50° C. with di-tert-butyl chloromethyl phosphate (Krise et al. 1999) using sodium hydride as a base and tetrabutylammonium iodide as an auxiliary, yielding the phosphorylated aldehyde 11. Subsequent reduction and silylation of the formed alcohol can lead to 12, which can be then transformed, analogously as described in Scheme III, into the mesylate 13.

If desired, the optically pure version of a salmeterol derivative can be obtained according to Schemes I and II, using a single, desired enantiomer prepared as described in literature (e.g. Hett et al., 1994).

An example of an alternative process for the synthesis of the optically pure, phosphorylated β-agonist with an alternate side chain is illustrated on Scheme V. The vinyl compound 7 is asymmetrically dihydroxylated using AD-mix-beta, producing diol 14. The selective tosylation proceeds on the primary hydroxyl, which is ensured by the presence of a catalytic amount of dibutyltin oxide, thus forming intermediate 15. The chiral epoxide 16 is obtained by brief and low-temperature treatment with sodium hexamethyldisilazide as a base. The opening of the epoxide with the amine of choice (bearing the R₄ moiety) can lead to aminoalcohol 17, which can be later transformed through manipulation of protective groups and final mesylation into an activated, chiral intermediate 18. If the whole synthetic sequence described above is applied to bromocompound 12 as a substrate, the final result can be the mesylate analog 19.

Scheme VI illustrates the convergent assembly of the mutual prodrugs of AISTM and β-agonist. The selected AISTM's (prepared according to literature procedures) are alkylated with the benzylic mesylate of the protected, phosphorylated β-agonist derivatives (3, 5, 10, 13, 18 or 19) in the presence of about a stoichiometric amount of sodium iodide in a polar, aprotic solvent like acetonitrile. In the final step, the intermediate quaternary ammonium salts are deprotected by mild acidolysis, either by brief (up to 1 hour) treatment with about 4N HCl in dioxane or in low-temperature treatment with TFA in dichloromethane at about 0° C., yielding the target mutual prodrugs of invention.

### II. Enzymatic Activation of Monophosphates as Mutual AISTM-β-Agonist Prodrugs

Monophosphates described in the compounds of Formula A (mutual prodrugs of AISTMs and β-agonists) are designed to release both drugs in a multistep bioactivation process. First, alkaline phosphatase present in lungs (in the case of topical delivery) efficiently dephosphorylates the mutual prodrug triggering a cascade of chemical breakdown/hydrolysis that can be combined with the subsequent enzymatic hydrolysis in the case of a double mutual prodrug (when an AISTM is additionally masked as an ester prodrug). It can be assumed that the phosphate cleavage is not a rate determining step, occurring faster relatively to the subsequent processes. The number of steps required and their respective kinetics depend on the structure of the mutual prodrug undergoing bioactivation. For example, if a methylenoxy-linker to a monophosphate moiety is present then the subsequent elimination of formaldehyde occurs at physiologic pH. Thus the phenolate intermediate forms, which is highly prone to spontaneous hydrolysis occurring at the benzylic position, which “restores” the saligenin moiety of a β-agonist. That step is likely rate-determining and it might be influenced by the steric and electronic nature of the “leaving group” R₁R₂R₃X. The departing moiety R₁R₂R₃X is either an AISTM itself, or its ester precursor, that in the final step of enzymatic cleavage by the nonspecific lung esterases delivers an AISTM at the desired site of its action.

The bioactivation described above is depicted on Scheme VII and the examples of such transformation are described in Examples 93 and 94 (in vitro and in vivo, respectively).

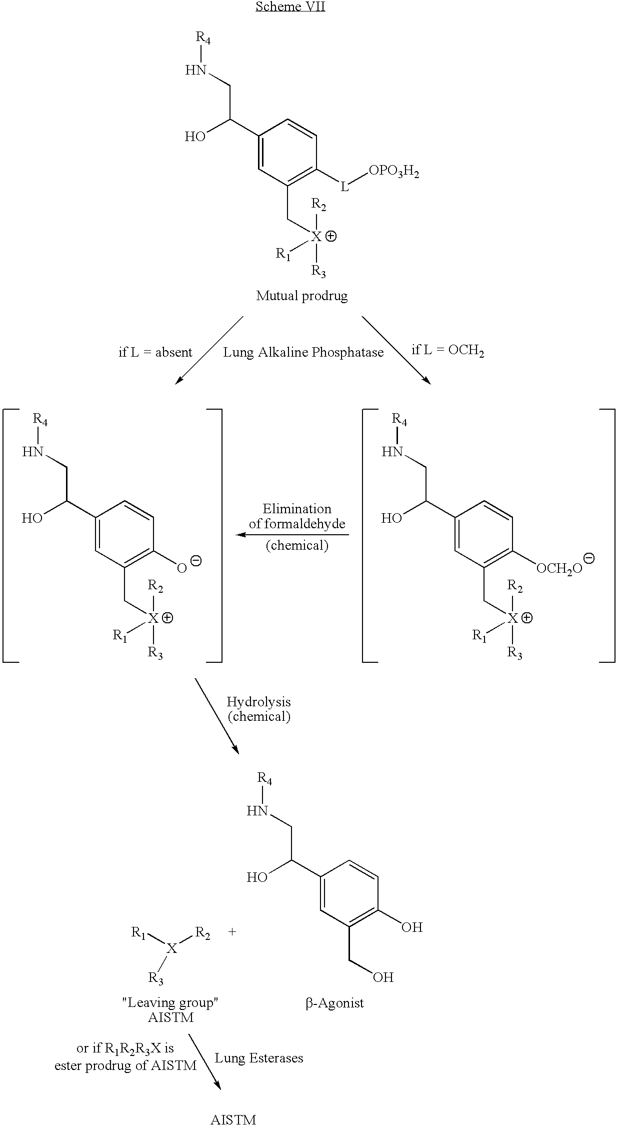

### III. Aerosol Delivery Devices

The use of the monophosphates of Formula A, suitably formulated for liquid nebulization or alternatively as a dry powder, provides a sufficient amount of the mutual prodrug to the lungs to achieve a therapeutic effect through the release of both bioactive components locally. Monophosphate mutual prodrugs of the invention are suitable for aerosolization using jet, electronic, or ultrasonic nebulizers. They are also appropriate for delivery by dry powder or metered dose inhaler. Their solid form has long-term stability permitting the drug substance to be stored at room temperature.

The aerosol formulation may comprise a concentrated solution of about 1-10 mg/mL of a compound of Formula A or its pharmaceutically acceptable salt, dissolved in aqueous or aqueous-ethanolic solution. Preferably the aerosol formulation has a pH between about 4.0 and about 7.5. Preferred pharmaceutically acceptable salts are inorganic acid salts including hydrochloride, hydrobromide, sulfate or phosphate salts as they may cause less pulmonary irritation. The therapeutic amount of the mutual prodrug of the present invention is delivered to the lung endobronchial space by nebulization of a liquid aerosol or dry powder having an average mass median diameter between about 1 to about 5μ. A liquid formulation may require separation of a mutual prodrug salt from the appropriate diluent requiring reconstitution prior to administration because the long-term stability of the monophosphate mutual prodrugs in aqueous solutions may not provide a commercially acceptable shelf life.

An indivisible part of this invention is a device able to generate aerosol from the formulation of the invention into aerosol particles predominantly in the about 1-5μ size range. Predominantly, in this application, means that at least about 70% but preferably more than about 90% of all generated aerosol particles are within the about 1-5μ size range. Typical devices include jet nebulizers, ultrasonic nebulizers, vibrating porous plate nebulizers, and energized dry powder inhalers.

A jet nebulizer utilizes air pressure to break a liquid solution into aerosol droplets. An ultrasonic nebulizer works by a piezoelectric crystal that shears a liquid into small aerosol droplets. A pressurized nebulization system forces solution under pressure through small pores to generate aerosol droplets. A vibrating porous plate device utilizes rapid vibration to shear a stream of liquid into appropriate droplet sizes. However, only some formulations of monophosphate mutual prodrugs can be efficiently nebulized, as the devices are sensitive to the physical and chemical properties of the formulation. Typically, the formulations which can be nebulized must contain small amounts of the monophosphate mutual prodrugs, which are delivered in small volumes (about 50-250 μL) of aerosol.

### IV. Utility

The compounds of the invention are useful (in humans) for treating pulmonary inflammation and bronchoconstriction.

The amount of active ingredient that may be combined with the carrier materials to produce a single dosage form will vary depending upon the host treated and the particular mode of administration.

This small volume, high concentration formulation of compounds of Formula A can be delivered as an aerosol and at efficacious concentrations to the respiratory tract in patients suffering from mild to severe asthma, chronic bronchitis or chronic obstructive pulmonary disease (COPD). The solid dosage formulation is stable, readily manufactured and very cost effective. Furthermore, the formulation provides adequate shelf life for commercial distribution. The mutual prodrug of the present invention masks the systemic side effects of AISTM's, like nausea, diarrhea, headaches or immune suppression. The mutual prodrug also masks the β-agonist activity minimizing a chance for cardiovascular side-effects. Both drugs are released by enzymes present in the lungs, specifically alkaline phosphatase, thereby releasing simultaneously the therapeutic amount of a β-agonist and of an AISTM, at the site of inflammation and bronchoconstriction.

The foregoing may be better understood from the following examples, which are presented for the purposes of illustration and are not intended to limit the scope of the inventive concepts.

### Example 1

### Phosphorobromidic acid di-tert-butyl ester

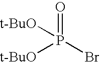

The title phosphorylating agent was prepared according to modified conditions compared to those described by Gajda and Zwierzak (1976). By lowering the temperature of the reaction to 15° C. and decreasing the reaction time to 2.5 hours the title compound obtained in our hands had better purity then when applying the literature conditions (25° C. for 4 hours). The title phosphobromidate is unstable and was immediately used for the phosphorylation reactions (see Examples 4 and 10).

Examples 2-6 illustrate the synthesis of the racemic phosphorylated derivative of salmeterol (see Scheme I).

### Example 2

### [2-Hydroxy-2-(4-hydroxy-3-hydroxymethyl-phenyl)-ethyl]-[6-(4-phenyl-butoxy)-hexyl-carbamic acid tert-butyl ester

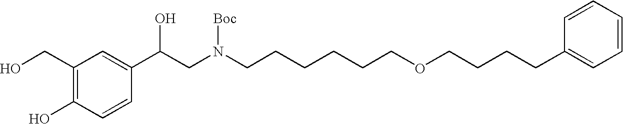

Commercially available salmeterol xinafoate (6.04 g, 10 mmol) and potassium carbonate (1.39 g, 10 mmol) were suspended with stirring in a 1,4-dioxane/water mixture (1:1, 80 mL). Then, di-t-butyl-dicarbonate (2.40 g, 11 mmol) dissolved in 1,4-dioxane (10 mL) was added dropwise while continuing stirring at room temperature. The TLC analysis after 30 minutes showed only traces of starting material. After 2 hours 1,4-dioxane was evaporated and the suspension formed was diluted with water and extracted twice with chloroform (125 mL total). Then, the organic layer was washed with saturated sodium bicarbonate, brine and dried over anhydrous magnesium sulfate. The crude material obtained after decantation and evaporation was purified by silica gel chromatography eluting with the ethyl acetate/hexane mixture (1:1). The title compound (4.61 g, 89%) was obtained as a glassy residue solidifying upon refrigeration.

LCMS: 100%, MNa⁺ 538.3 (exact mass 515.3 calcd for C₃₀H₄₅NO₆). Anal. Calc: C, 69.87; H, 8.80; N, 2.72. Found: C, 69.69; H, 8.64; N, 2.68.

### Example 3

### [2-(3-Formyl-4-hydroxy-phenyl)-2-hydroxy-ethyl]-[6-(4-phenyl-butoxy)-hexyl]-carbamic acid tert-butyl ester

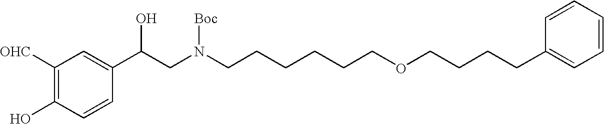

The N-Boc-salmeterol described in Example 2 (3.24 g, 6.28 mmol) was dissolved in chloroform (50 mL) and the activated manganese oxide (IV) (6.44 g, 85% w/w, 63 mmol) was added in portions with vigorous stirring. After 24 hours at room temperature the slurry was filtered through a pad of Celite, followed by the concentration of the filtrate combined with the chloroform washes. The crude residue thus obtained was purified by silica gel chromatography using ethyl acetate/hexane mixture (1:5) yielding the title aldehyde 1 (2.45 g, 77%). LCMS: 96%, MNa⁺ 536.3 (exact mass 513.3 calcd for C₃₀H₄₃NO₆).

### Example 4

### {2-[4-(Di-tert-butoxy-phosphoryloxy)-3-formyl-phenyl]-2-hydroxy-ethyl}-[6-(4-phenyl-butoxy)-hexyl]-carbamic acid tert-butyl ester

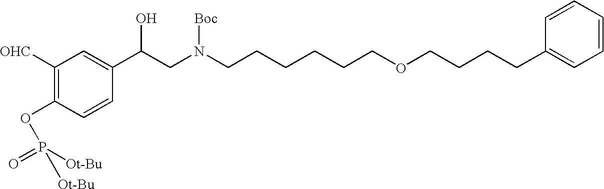

Aldehyde 1 (3.44 g, 6.69 mmol) was dissolved in anhydrous THF (10 mL), which was followed by adding DMAP (82 mg, 0.67 mmol) and DBU (1.11 mL, 7.4 mmol) with vigorous stirring under nitrogen. After cooling the reaction mixture to 0° C. the phosphobromidate described in Example 1 (2.19 g, 8 mmol) diluted with anhydrous THF (5 mL) was added dropwise over 15 minutes Stirring under nitrogen at 0° C. was continued for another 30 minutes, after which the TLC analysis showed the phosphorylation to be almost complete. After another 60 minutes the reaction mixture was concentrated, the residue was redissolved in ethyl acetate, washed 3 times with 10% citric acid, twice with 0.5N NaOH, brine and dried over anhydrous sodium sulfate. The organic phase was then filtered through a pad of basic alumina and the filtrate combined with ethyl acetate washes was concentrated in vacuo. The crude product was purified by silica gel chromatography using 30% ethyl acetate/1% triethylamine in hexane, yielding the title compound 2 (3.42 g, 72%) as a glassy residue.

³¹PNMR (CDCl₃): −15.107 ppm. LCMS: 100%, MNa⁺ 728.0 (exact mass 705.4 calcd for C₃₈H₆₀NO₉P). Anal. Calc: C, 64.66; H, 8.57; N, 1.98. Found: C, 64.09; H, 8.54; N, 2.02.

### Example 5

### {2-[4-(Di-tert-butoxy-phosphoryloxy)-3-hydroxymethyl-phenyl]-2-hydroxy-ethyl}-[6-(4-phenyl-butoxy)-hexyl]-carbamic acid tert-butyl ester

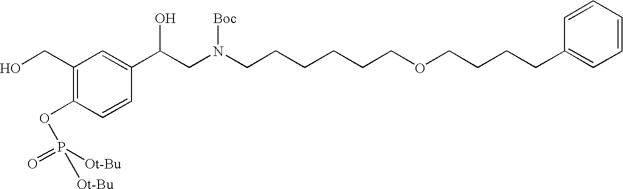

The phosphorylated aldehyde 2 (2.68, 3.8 mmol) was dissolved in anhydrous THF (10 mL) and the mixture was cooled to −78° C. Then, solid sodium borohydride (0.432 g, 11.4 mmol) was added in portions over 5 minutes with vigorous stirring under nitrogen, which was followed by adding methanol (1 mL). The reaction mixture was stirred allowing the temperature of the bath to increase to 0° C. over 4 hours (during which the TLC analysis showed consumption of the starting material). The reaction mixture was diluted with dichloromethane (50 mL), followed by careful quenching by adding 10% citric acid (20 mL) with vigorous stirring. The organic phase was separated, aqueous layer extracted with another portion of DCM and combined extracts were washed twice with saturated bicarbonate, brine, dried over anhydrous sodium sulfate, decanted and evaporated. The crude product was purified by chromatography using 40% ethyl acetate/1% triethylamine in hexane, yielding the title diol (2.01 g, 75%) as a colorless glassy residue.

¹H NMR (CDCl₃) selected signals: 7.17-7.41 (m, 8H), 4.92 (m, 1H), 4.62 (bs, 2H), 3.39 (q, 2H), 2.64 (t 2H), 1.62 (m, 4H), 1.54 (s, 9H), 1.52 (s, 9H), 1.49 (s, 9H), 1.115-1.49 (m, 8H). ³¹PNMR (CDCl₃): −13.060 ppm. LCMS: 99%, MNa⁺ 730.0 (exact mass 707.4 calcd for C₃₈H₆₂NO₉P). Anal. Calc: C, 64.48; H, 8.83; N, 1.98. Found: C, 64.70; H, 8.84; N, 1.90.

### Example 6

### Methanesulfonic acid 5-(2-{tert-butoxycarbonyl-[6-(4-phenyl-butoxy)-hexyl]-amino}-1-hydroxy-ethyl)-2-(di-tert-butoxy-phosphoryloxy)-benzyl ester (3)

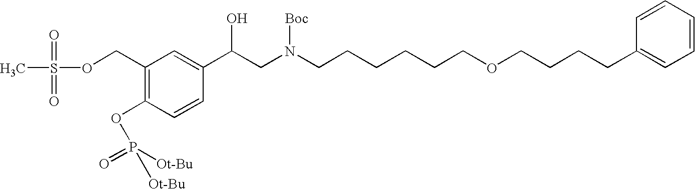

Compound 3 was synthesized by treating the diol described in Example 5 dissolved in anhydrous dichloromethane at 0° C. with the 1.1 equivalent of methanesulfonyl chloride in presence of 2 equiv. of 1,2,2,6,6-pentamethyl-piperidine (PMP). The TLC monitoring showed the disappearance of the starting material after 15-30 minutes. After 1 hour the reaction mixture was concentrated in vacuo, redissolved in ethyl acetate, washed with 10% citric acid solution, saturated bicarbonate solution, brine, dried over anhydrous magnesium sulfate, decanted and evaporated. Thus obtained mesylate 3 was directly used for the quaternization (alkylation) of the MRA molecules (see Scheme VI).

Examples 7-9 illustrate the synthesis of the phosphonooxy-methylene derivative of salmeterol.

### Example 7

### {2-[4-(Di-tert-butoxy-phosphoryloxymethoxy)-3-formyl-phenyl]-2-hydroxy-ethyl}-[6-(4-phenyl-butoxy)-hexyl]-carbamic acid tert-butyl ester

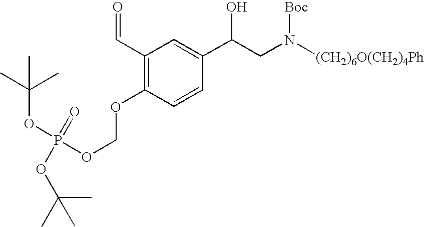

Salmeterol derivative 1 was alkylated with (t-BuO)₂P═O(OCH₂Cl) (1.2 equivalent added in portions—judges by TLC) according to the procedure analogous to the publication by Krise et al. (1999). Sodium hydride was used as a base (1 equivalent) and TBAI as a catalyst (0.2 equiv.) and the reaction was carried out in anhydrous THF with gentle heating (50° C.). Overall reaction time to consume the starting material was 18 hours, after which the mixture was cooled to room temperature and quenched with 10% (w/v) aqueous citric acid followed by THF removal via rotary evaporatoration. The resulting mixture was extracted with diethyl ether (twice), the organic extracts were combined, and washed with: 0.5 M NaOH (3 times), 10% (w/v) aqueous citric acid, deionized water and brine, dried over anhydrous sodium sulfate and concentrated to yield crude 98% of brown, oily residue. That material was purified by silica gel chromatography, using the gradient (hexane/ethyl acetate—with both solvents buffered with 1% triethyl amine) to yield 70% of a clear, viscous oil.

LC-MS MNa⁺=758 observed; HPLC with UV detector at 272 nm: 95 area %; ³¹P NMR in DMSO-d6: −10.892 ppm.

### Example 8

### {2-[4-(Di-tert-butoxy-phosphoryloxymethoxy)-3-hydroxymethyl-phenyl]-2-hydroxy-ethyl}-[6-(4-phenyl-butoxy)-hexyl]-carbamic acid tert-butyl ester

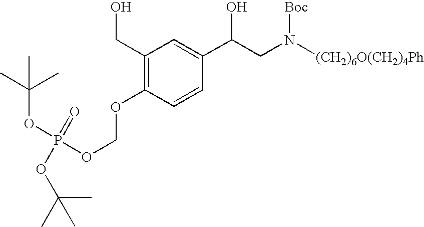

Aldehyde 4 was reduced analogously as described in Example 5, yielding the title compound in 92% yield of a slightly yellowish, viscous oil. LC-MS: MNa+=760 observed; HPLC at 272 nm: 96%. ³¹P NMR in DMSO-d6: −11.104 ppm.

### Example 9

### Methanesulfonic acid 5-(2-{tert-butoxycarbonyl-[6-(4-phenyl-butoxy)-hexyl]-amino}-1-hydroxy-ethyl)-2-(di-tert-butoxy-phosphoryloxymethoxy)-benzyl ester

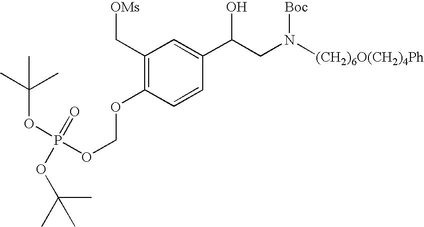

The diol described in Example 8 was selectively mesylated according to the procedure described in Example 6, yielding the mesylate 5 in high yield, which was used directly for quaternization reactions.

Examples 10-17 illustrate the synthesis of the racemic phosphorylated derivative of albuterol (see Scheme III).

### Example 10

### Phosphoric acid 4-bromo-2-formyl-phenyl ester di-tert-butyl ester

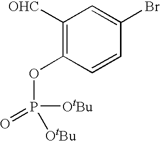

5-Bromosalicylaldehyde (8.04 g, 40 mmol) was phosphorylated analogously as described in Example 4, using DBU (6.58 mL, 44 mmol) and DMAP (0.489 g, 4 mmol) dissolved in anhydrous THF (50 mL) and cooled to 0° C. The phosphorylating agent was prepared as described in Example 1 (23.2 g, 85 mmol) and diluted with anhydrous THF (20 mL). The crude product was purified by chromatography (9% ethyl acetate+1% triethylamine in hexane) yielding analytically pure title aldehyde 6 as a yellowish solid (11.51 g, 73%).

¹HNMR (CDCl₃): 10.35 (s, 1H), 7.99 (d, 1H, J=2.4 Hz), 7.67 (dd, 1H, J=8.8 Hz, 2.4 Hz), 7.41 (d, 1H, J=8.8 Hz), 1.51 (s, 18H). ³¹PNMR (CDCl₃): −15.239 ppm. LCMS: 99%, MNa⁺ 415 (exact mass 392.04 calcd for C₁₅H₂₂BrO₅P).

### Example 11

### Phosphoric acid 4-bromo-2-(tert-butyl-dimethyl-silanyloxymethyl)-phenyl ester di-tert-butyl ester

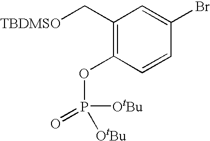

Aldehyde described in Example 10 was reduced to alcohol analogously as described in Example 5. The crude material solidified upon repeated evaporation with hexane and was sufficiently pure to continue the synthesis. The intermediate alcohol was converted to compound 6 by treatment with the slight excess of tert-butyldimethylsilyl chloride in DMF in presence of excess (5 equivalents) of imidazole. After the overnight reaction at room temperature the mixture was diluted with diethyl ether, washed extensively with 10% citric acid, brine and the organic phase was then dried with anhydrous magnesium sulfate, decanted and evaporated. The crude material was purified by chromatography using 10% ethyl acetate+1% triethylamine in hexane.

### Example 12

### Phosphoric acid di-tert-butyl ester 2-(tert-butyl-dimethyl-silanyloxymethyl)-4-vinyl-phenyl ester

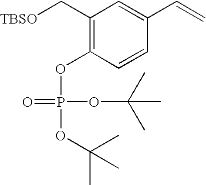

A two-neck, round bottomed flask, equipped with a reflux condenser was charged with the solution of compound 6 in a mixture of toluene (8 mL/mmol) and ethanol (1 mL/mmol) followed by adding a degassed 20% solution of potassium carbonate (8 mL/mmol). The biphasic mixture was vigorously stirred for 1 hour while the stream of argon was passed through the flask. To this mixture, the trivinylboroxine-pyridine complex (1.5 equivalents) was added, followed by tricyclohexylphosphine (0.1 equivalent). The reaction mixture purged with argon once again for 30 minutes, then palladium (II) acetate (0.1 equivalents) was added, followed by vigorous stirring and heating under reflux under the positive pressure of argon for 4 hours. After that time TLC analysis (chloroform/methanol 8:1) showed the complete consumption of starting material. The reaction mixture was diluted with ethyl acetate (3 times the original volume) and the organic phase was washed with water (3 times), 10% citric acid solution (twice) and brine and was dried over anhydrous MgSO₄. After filtration and evaporation of the solvent, the residue was purified by silica gel chromatography (ethyl acetate/hexanes 1:20 with 5% of triethylamine), yielding 80% of the desired olefin 7 as a viscous oil.

¹H NMR (CDCl₃): 7.52 (s, 1H), 7.27 (d, 1H), 7.19 (d, 1H), 6.67 (dd, 1H), 5.66 (d, 1H), 5.17 (d, 1H), 4.71 (s, 2H), 1.48 (s, 18H), 0.95 (s, 9H), 0.10 (s, 6H). ³¹P NMR (CDCl₃): −14.18 ppm. LCMS: 95%, MNa+ 479 (exact mass 456.3 calcd for C₂₃H₄₁O₅PSi).

### Example 13

### Phosphoric acid di-tert-butyl ester 2-(tert-butyl-dimethyl-silanyloxymethyl)-4-oxiranyl-phenyl ester

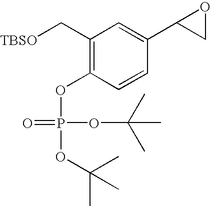

Oxone® (8 g, 13.1 mmol) was slowly added to a stirring solution of compound 7 (1.2 g, 2.63 mmol) in a CH₂Cl₂/satd NaHCO₃ mixture (20 mL, 3:5) and acetone (10 mL) at 0° C. The pH of the mixture was adjusted to >7.5 with satd NaHCO₃ as needed. After stirring for 30 minutes. at 0° C. then 90 minutes at room temperature the resulting suspension was extracted with CH₂Cl₂ (3×15 mL), dried over Na₂SO₄ and concentrated to give crude epoxide (1.3 g) as light yellow oil. Chromatography (3:1 hexanes/ethyl acetate, 0.5% Et₃N) afforded the title epoxide (0.804 g, 65%) as clear oil: ¹H NMR (400 MHz, DMSO-D6) δ 7.36 (s, 1H), 7.23 (m, 2H), 4.74 (s, 2H), 3.92 (dd, 1H, J=2.6, 4.1), 3.11 (dd, 11H, J=4.1, 5.3), 2.77 (dd, 1H, J=2.6, 5.3), 1.43 (s, 18H), 0.90 (s, 9H), 0.08 (s, 6H).

### Example 14

### Phosphoric acid di-tert-butyl ester 4-(2-tert-butylamino-1-hydroxy-ethyl)-2-(tert-butyl-dimethyl-silanyloxymethyl)-phenyl ester

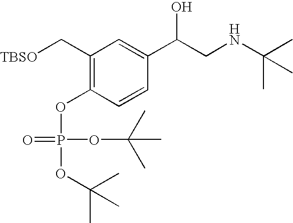

Solid LiClO₄ (180 mg, 1.7 mmol) was added to a stirring solution of epoxide described in Example 13 (4 g, 8.5 mmol) in tert-butylamine (9 mL, 84 mmol) at while stirring at room temperature. The resulting mixture was stirred for 48 hours, and then diluted with ethyl acetate (20 mL). The organic layer was washed with water, brine, dried over Na₂SO₄ and concentrated to give crude aminoalcohol (5.3 g) as yellow oil. Chromatography (9:1, CH₂Cl₂/MeOH, 0.5% Et₃N) afforded the title compound 8 (4.2 g, 91%) as light yellow oil.

¹H NMR (400 MHz, DMSO-D6) δ 7.45 (s, 1H), 7.23 (dd, 1H, J=2.1, 8.4), 7.18 (d, 1H, J=9.0), 4.75 (s, 2H), 4.49 (t, 1H, J=6.2), 3.17 (s, 1H), 2.58 (d, 2H, J=6.3), 1.42 (m, 18H), 1.01 (d, 9H, J=14.4), 0.92 (s, 9H), 0.06 (s, 6H); ES/MS, calcd for C₂₇H₅₃NO₆PSi 546.34, found m/z=546.4 (M+H).

### Example 15

### Carbonic acid tert-butyl ester 2-tert-butylamino-1-[3-(tert-butyl-dimethyl-silanyloxymethyl)-4-(di-tert-butoxy-phosphoryloxy)-phenyl]-ethyl ester

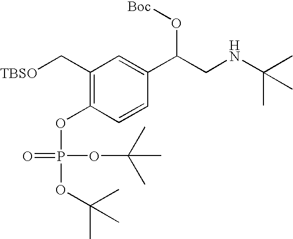

Solid (Boc)₂O (1.04 g, 4.79 mmol) was added to a stirred solution of 8 (1.74 g, 3.19 mmol), PMP (1.7 mL, 9.6 mmol), and DMAP (39 mg, 0.319 mmol) in anhydrous CH₃CN (30 mL) at 0° C. After 90 minutes the resulting mixture was quenched with saturated NaHCO₃ (40 mL) and extracted with ethyl acetate (3×30 mL). The combined organic layers were washed with brine, dried over Na₂SO₄, and concentrated to give crude carbonate (2.93 g) as white solid. Chromatography (1:3, hexanes/ethyl acetate, 0.5% Et₃N) afforded the title compound 9 (0.946 g, 46%) as clear oil.

¹H NMR (400 MHz, DMSO-D6) δ 7.43 (s, 1H), 7.23 (m, 2H), 5.38 (dd, 1H, J=5.0, 7.7), 4.75 (s, 2H), 2.79 (m, 2H), 1.43 (s, 18H), 1.36 (s, 9H), 0.96 (s, 9H), 0.92 (s, 9H), 0.07 (m, 6H); ES/MS, calcd for C₃₂H₆₁NO₈PSi 646.39, found m/z=646.5 (M+H).

### Example 16

### Carbonic acid tert-butyl ester 2-tert-butylamino-1-[4-(di-tert-butoxy-phosphoryloxy)-3-hydroxymethyl-phenyl]-ethyl ester

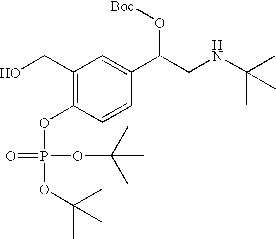

A 1.0M solution of TBAF in THF (1.4 mL, 1.4 mmol) was added to a stirred solution of compound 9 (0.9 g, 1.4 mmol) in anhydrous THF (14 mL) at room temperature. The resulting suspension was stirred for 1 hour, then quenched with satd NaHCO₃ (20 mL) and the aqueous layer was extracted with ethyl acetate (3×20 mL). The combined organic layers were washed with brine, dried over Na₂SO₄, and concentrated to give crude alcohol (1.01 g) as light yellow oil. Chromatography (1:3, hexanes/ethyl acetate, 0.5% Et₃N) afforded pure title compound (0.61 g, 82%) as a clear oil.

¹H NMR (400 MHz, DMSO-D6) δ 7.45 (s, 1H), 7.21 (m, 2H), 5.40 (dd, 1H, J=4.8, 8.0), 5.22 (t, 1H, J=5.6), 4.56 (d, 2H, J=5.5), 2.79 (ddd, 2H, J=6.5, 12.3, 17.1), 1.43 (m, 18H), 1.37 (s, 9H), 0.98 (s, 9H); ES/MS, calcd for C₂₆H₄₇NO₈P 532.30, found m/z=532.4 (M+H).

### Example 17

### Methanesulfonic acid 5-[2-(tert-butoxycarbonyl-tert-butyl-amino)-1-hydroxy-ethyl]-2-(di-tert-butoxy-phosphoryloxy)-benzyl ester

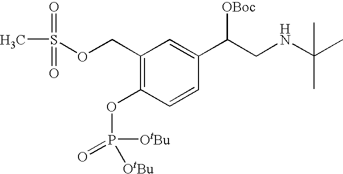

A solution of methanesulphonyl chloride (105 L, 1.36 mmol) in CH₂Cl₂ (0.5 mL) was added dropwise to a stirred solution of compound described in Example 16 (0.6 g, 1.13 mmol) and PMP (817 L, 4.52 mmol) in CH₂Cl₂ (12 mL) at 0° C. The reaction mixture was stirred for 30 minutes then quenched with satd NaHCO₃ (20 mL). The organic layer was separated, dried over Na₂SO₄, and concentrated to give crude mesylate (0.98 g) as light yellow oil. Chromatography (1:3, hexanes/ethyl acetate, 0.5% Et₃N) afforded the title mesylate 10 (0.56 g, 76%) as a clear oil. ES/MS, calcd for C₂₇H₄₉NO₁₀PS 610.28, found m/z=610.4 (M+H).

Examples 18-25 illustrate the synthesis of phosphonooxy-methylene derivative of racemic albuterol (salbutamol).

### Example 18

### Phosphoric acid 4-bromo-2-formyl-phenoxymethyl ester di-tert-butyl ester

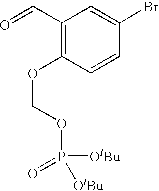

The title compound 11 can be synthesized analogously as described in Example 7, using the 5-bromosalicaldehyde as a starting material.

### Example 19

### Phosphoric acid 4-bromo-2-(tert-butyl-dimethyl-silanyloxymethyl)-phenoxymethyl ester di-tert-butyl ester

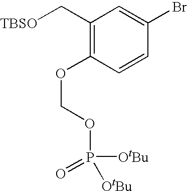

The title compound 12 can be synthesized analogously as described in Example 11, using the aldehyde 11 as a starting material.

### Example 20

### Phosphoric acid di-tert-butyl ester 2-(tert-butyl-dimethyl-silanyloxymethyl)-4-vinyl-phenoxymethyl ester

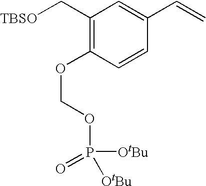

The title compound can be synthesized by the Suzuki vinylation described in Example 12, using the bromocompound 12 as a starting material.

### Example 21

### Phosphoric acid di-tert-butyl ester 2-(tert-butyl-dimethyl-silanyloxymethyl)-4-oxiranyl-phenoxymethyl ester

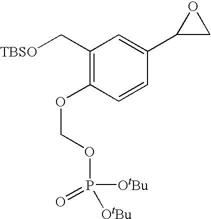

The title compound can be synthesized through epoxidation described in Example 13, using the compound described in Example 20 as a starting material.

### Example 22

### Phosphoric acid di-tert-butyl ester 4-(2-tert-butylamino-1-hydroxy-ethyl)-2-(tert-butyl-dimethyl-silanyloxymethyl)-phenoxymethyl ester

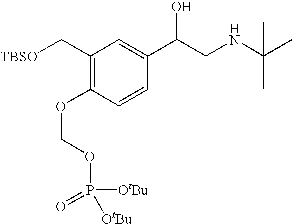

The aminolysis with t-butylamine (as described in Example 14) can be used to synthesize the compound depicted above using compound from Example 21 as a substrate.

### Example 23

### Carbonic acid tert-butyl ester 2-tert-butylamino-1-[3-(tert-butyl-dimethyl-silanyloxymethyl)-4-(di-tert-butoxy-phosphoryloxymethoxy)-phenyl]-ethyl ester

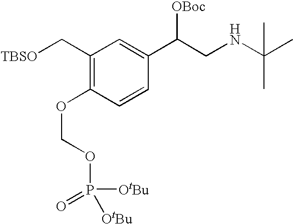

The O-acylation (protection) of the aminoalcohol described in Example 22 can be accomplished according to the procedure described in Example 15.

### Example 24

### Carbonic acid tert-butyl ester 2-tert-butylamino-[4-(di-tert-butoxy-phosphoryloxymethoxy)-3-hydroxymethyl-phenyl]-ethyl ester

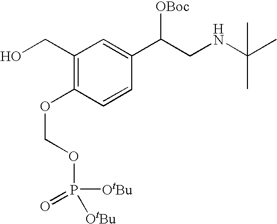

The TBS-removal from compound described in the previous Example can be achieved analogously as described in Example 16.

### Example 25

### Methanesulfonic acid 5-(1-tert-butoxycarbonyloxy-2-tert-butylamino-ethyl)-2-(di-tert-butoxy-phosphoryloxymethoxy)-benzyl ester

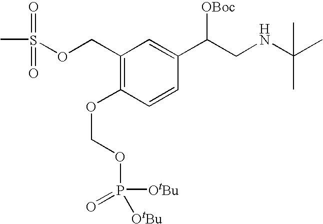

Title compound 13 can be synthesized according to the procedure described in Example 17, using the aminoalcohol from Example 24 as a substrate.

Examples 26-28 illustrate the synthesis of the asymmetric intermediate, that can be used to prepare optically pure β-agonist derivatives (see Scheme V).

### Example 26

### Phosphoric acid di-tert-butyl ester 2-(tert-butyl-dimethyl-silanyloxymethyl)-4-(1,2S-dihydroxy-ethyl)-phenyl ester

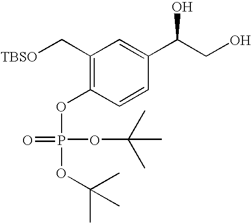

A solid AD-mix β reagent (300 mg) was added to a stirred solution of 7 (100 mg, 0.219 mmol) in t-BuOH (1 mL) and H₂O (1 mL) at 0° C. After stirring for 19 hours solid Na₂SO₃ (300 mg) was added to quench and the resulting reaction mixture was allowed to warm up to room temperature and stirred for additional 1 hour. After being diluted with water the reaction mixture was extracted with CH₂Cl₂ (3×15 mL). The combined organic layers were dried over Na₂SO₄ and concentrated to give crude diol (123 mg) as pale yellow oil. Chromatography (1:3, hexanes/ethyl acetate, 0.5% Et₃N) afforded title compound 14 (93 mg, 87%) as clear oil.

¹H NMR (400 MHz, DMSO-D6) δ 7.46 (d, 1H, J=8.4 Hz), 7.18 (m, 2H), 5.20 (brd, 2H, J=48.0 Hz), 4.53 (m, 3H), 3.41 (d, 2H, J=6.7 Hz), 1.43 (s, 18H), 0.83 (s, 6H), −0.06 (s, 6H); ES/MS calcd for C₂₃H₄₃NaO₇PSi 513.24, found m/z=513.3 (M+Na).

### Example 27

### Toluene-4-sulfonic acid 2-[3-(tert-butyl-dimethyl-silanyloxymethyl)-4-(di-tert-butoxy-phosphoryloxy)-phenyl]-2S-hydroxy-ethyl ester

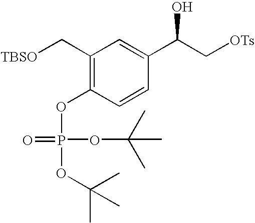

To a stirred solution of compound 14 (660 mg, 1.35 mmol) in CH₂Cl₂ (13 mL) dibutyltinoxide (0.7 mg, 0.0027 mmol), Et₃N (188 μL, 1.35 mmol), and TsCl (257 mg, 1.35 mmol) were added in the aforementioned order at room temperature. The reaction mixture was stirred for 90 minutes and then quenched with H₂O (20 mL). The aqueous layer was extracted with CH₂Cl₂ (3×15 mL). The combined organic layers were dried over Na₂SO₄ and concentrated to give crude monotosylate (1.19 g) as opaque semi solid. Chromatography (1:1, hexanes/ethyl acetate, 0.5% Et₃N) afforded pure 15 (700 mg, 81%) as clear oil.

¹H NMR (400 MHz, DMSO-D6) δ 7.67 (m, 2H), 7.43 (m, 2H), 7.36 (s, 1H), 7.18 (m, 2H), 5.80 (d, 1H, J=4.6 Hz), 4.76 (dd, 1H, J=5.3, 10.3 Hz), 4.71 (s, 2H), 3.95 (d, 2H, J=6.1 Hz), 2.40 (s, 3H), 1.43 (s, 18H), 0.89 (m, 9H), 0.05 (d, 6H, J=0.6 Hz); ES/MS calcd for C₃₀H₄₉NaO₉PSSi 667.25, found m/z=667.2 (M+Na).

### Example 28

### Phosphoric acid di-tert-butyl ester 2-(tert-butyl-dimethyl-silanyloxymethyl)-(S)-4-oxiranyl-phenyl ester

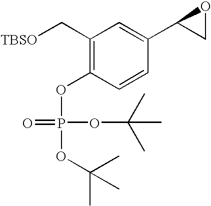

A 1.0M solution of NaHMDS in THF (1.3 mL, 1.30 mmol) was added dropwise to a stirred solution of 15 (420 mg, 0.651 mmol) in THF (7 mL) at 0° C. The resulting mixture was stirred for additional 10 minutes, quenched with satd NaHCO₃ (15 mL) and extracted with ethyl acetate (3×20 mL). The combined organic layers were washed with brine, dried over Na₂SO₄, and concentrated to give crude epoxide (293 mg) as pale yellow semi solid. Chromatography (3:1, hexanes/ethyl acetate, 0.5% Et₃N) afforded title compound 16 (250 mg, 81%) as clear oil.

¹H NMR (400 MHz, DMSO-D6) δ 7.36 (s, 1H), 7.23 (d, 2H, J=1.2 Hz), 4.74 (s, 2H), 3.93 (dd, 1H, J=2.6, 4.1 Hz), 3.11 (dd, 1H, J=4.1, 5.3 Hz), 2.78 (dd, 1H, J=2.6, 5.3 Hz), 1.41 (d, 18H, J=15.4 Hz), 0.90 (m, 9H), 0.06 (m, 6H).

Examples 29-92 illustrate the mutual prodrugs of AISTM's and beta-agonists, prepared according to Scheme VI.

### Example 29

### (2-{[5-(2,4-Difluoro-phenoxy)-1-isobutyl-1H-indazole-6-carbonyl]-amino}-ethyl)-(5-{-hydroxy-2-[6-(4-phenyl-butoxy)-hexylamino]-ethyl}-2-phosphonooxy-benzyl)-dimethyl-ammonium

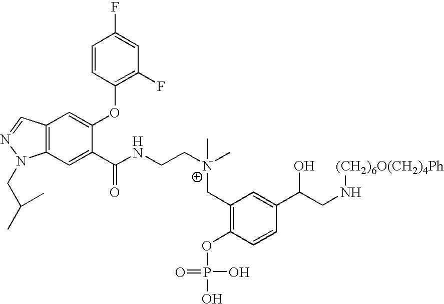

5-(2,4-Difluoro-phenoxy)-1-isobutyl-1H-indazole-6-carboxylic acid (2-dimethylamino-ethyl)-amide (ARRY-797; Munson et al., 2004) was converted to the title mutual prodrug through a two-step procedure as follows:

Quaternization step. Solid NaI (8 mg, 0.058 mmol) was added to a stirring solution of 5-(2,4-difluoro-phenoxy)-1-isobutyl-1H-indazole-6-carboxylic acid (2-dimethylamino-ethyl)-amide (81 mg, 0.195 mmol) and mesylate 3 (230 mg, 0.292 mmol) in anhydrous CH₃CN (4 mL) at room temperature. After stirring for 4 days the resulting suspension was concentrated to give crude quaternary salt. Chromatography (9:1, CH₂Cl₂/MeOH) afforded fractions of fully protected quaternary ammonium salt, as well as mono-t-butyl-phosphate, which were combined for the deprotection step.

ES/MS, calcd for C₂₂H₂₇F₂N₄O₂ 1106.62, found m/z=1106.7 (M⁺).

Deprotection and final purification step. A solution of 4N HCl in dioxane (1.5 mL) was added to a stirred solution of the protected quaternary ammonium salt (100 mg) in anhydrous CH₂Cl₂ (3 mL) at room temperature. After stirring for 1 hour ether (30 mL) was added and the mixture was stirred for additional 1 hour and then filtered. The filter cake was washed with ether (2×20 mL) and dried to give the title mutual prodrug (52 mg) in high enough purity as white solid. If necessary the compound could be further purified by the reverse-phase chromatography.

³¹P NMR (400 MHz, DMSO-d6) δ −5.4 ppm; ES/MS, calcd for C₄₇H₆₃F₂N₅O₅P⁺ 894.44, found min/z=894.5 (M+H).

### Example 30

### [5-(2-tert-Butylamino-1-hydroxy-ethyl)-2-phosphonooxy-benzyl]-(2-{[5-(2,4-difluoro-phenoxy)-1-isobutyl-1H-indazole-6-carbonyl]-amino}-ethyl)-dimethyl-ammonium

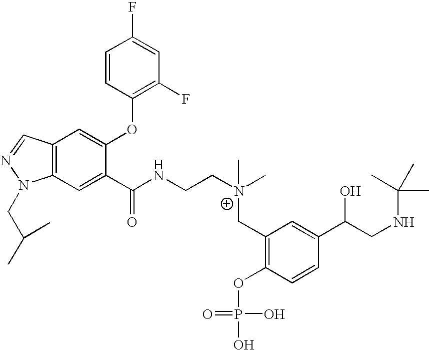

The title compound can be prepared by a two-step procedure described in Example 29, using 5-(2,4-Difluoro-phenoxy)-1-isobutyl-1H-indazole-6-carboxylic acid (2-dimethylamino-ethyl)-amide and mesylate 10 as starting materials.

### Example 31

### (2-{[5-(2,4-Difluoro-phenoxy)-1-isobutyl-1H-indazole-6-carbonyl]-amino}-ethyl)-(5-{1-hydroxy-2-[6-(4-phenyl-butoxy)-hexylamino]-ethyl}-2-phosphonooxymethoxy-benzyl)-dimethyl-ammonium

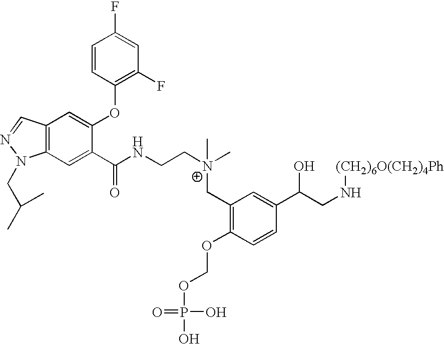

The title compound can be prepared by a two-step procedure described in Example 29, using 5-(2,4-Difluoro-phenoxy)-1-isobutyl-1H-indazole-6-carboxylic acid (2-dimethylamino-ethyl)-amide and mesylate 5 as starting materials, except that a TFA/DCM (1:1) mixture is used for a final deprotection carried out at 0° C. for 30 min.

### Example 32

### [5-(2-tert-Butylamino-1-hydroxy-ethyl)-2-phosphonooxymethoxy-benzyl]-(2-{[5-(2,4-difluoro-phenoxy)-1-isobutyl-1H-indazole-6-carbonyl]-amino}-ethyl)-dimethyl-ammonium

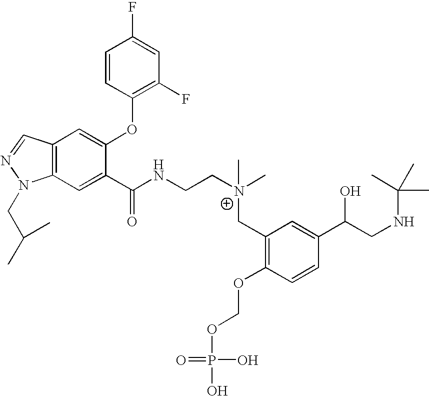

The title compound can be prepared by a two-step procedure described in Example 29, using 5-(2,4-Difluoro-phenoxy)-1-isobutyl-1H-indazole-6-carboxylic acid (2-dimethylamino-ethyl)-amide and mesylate 13 as starting materials, except that a TFA/DCM (1:1) mixture is used for a final deprotection carried out at 0° C. for 30 minutes.

### Example 33

### 3,5-Dichloro-4-(3-cyclopropylmethoxy-4-difluoromethoxy-benzoylamino)-1-(4-{1-hydroxy-2-[6-(4-phenyl-butoxy)-hexylamino]-ethyl}-2-phosphonooxy-benzyl)-pyridinium

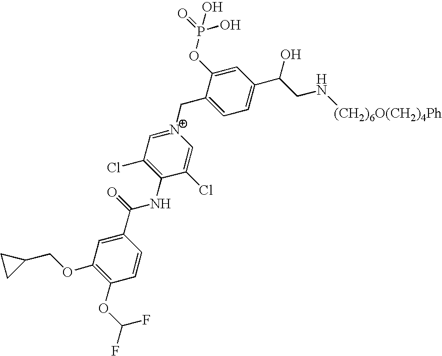

The title compound can be prepared by a two-step procedure described in Example 29, using the 3-cyclopropylmethoxy-N-(3,5-dichloro-pyridin-4-yl)-4-difluoromethoxy-benzamide (Roflumilast) and mesylate 3 as starting materials.

### Example 34

### 1-[4-(2-tert-Butylamino-1-hydroxy-ethyl)-2-phosphonooxy-benzyl]-3,5-dichloro-4-(3-cyclopropylmethoxy-4-difluoromethoxy-benzoylamino)-pyridinium

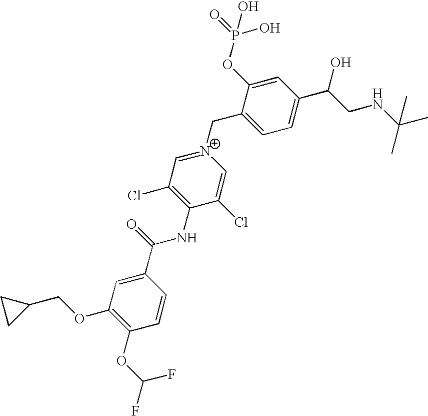

The title compound can be prepared by a two-step procedure described in Example 29, using the 3-cyclopropylmethoxy-N-(3,5-dichloro-pyridin-4-yl)-4-difluoromethoxy-benzamide (Roflumilast) and mesylate 10 as starting materials.

### Example 35

### 3,5-Dichloro-4-(3-cyclopropylmethoxy-4-difluoromethoxy-benzoylamino)-1-(4-{1-hydroxy-2-[6-(4-phenyl-butoxy)-hexylamino]-ethyl}-2-phosphonooxymethoxy-benzyl)-pyridinium

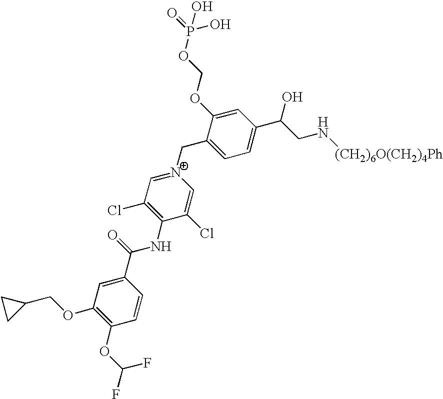

The title compound can be prepared by a two-step procedure described in Example 29, using the 3-cyclopropylmethoxy-N-(3,5-dichloro-pyridin-4-yl)-4-difluoromethoxy-benzamide (Roflumilast) and mesylate 5 as starting materials, except that a TFA/DCM (1:1) mixture is used for a final deprotection carried out at 0° C. for 30 minutes.

### Example 36

### 1-[4-(2-tert-Butylamino-1-hydroxy-ethyl)-2-phosphonooxymethoxy-benzyl]-3,5-dichloro-4-(3-cyclopropylmethoxy-4-difluoromethoxy-benzoylamino)-pyridinium

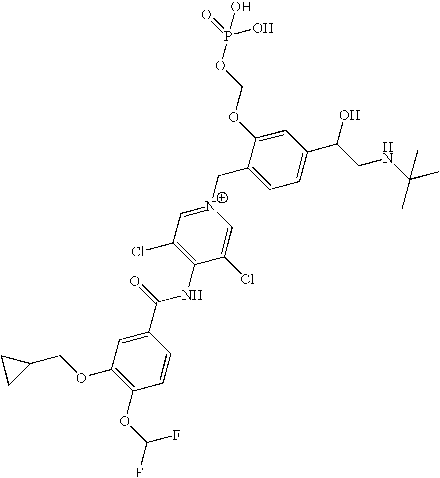

The title compound can be prepared by a two-step procedure described in Example 29, using the 3-cyclopropylmethoxy-N-(3,5-dichloro-pyridin-4-yl)-4-difluoromethoxy-benzamide (Roflumilast) and mesylate 13 as starting materials, except that a TFA/DCM (1:1) mixture is used for a final deprotection carried out at 0° C. for 30 minutes.

### Example 37

### 4-[2-(3-Cyclopentyloxy-4-methoxy-phenyl)-2-phenyl-ethyl]-1-(4-{1-hydroxy-2-[6-(4-phenyl-butoxy)-hexylamino]-ethyl}-2-phosphonooxy-benzyl)-pyridinium

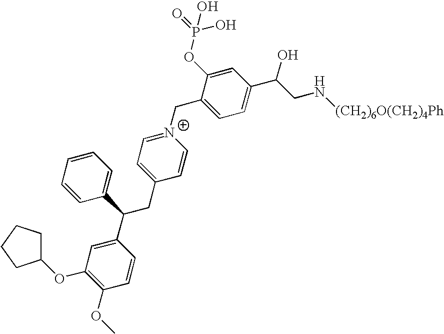

Quaternization step. To a solution of 4-[2-(3-cyclopentyloxy-4-methoxy-phenyl)-(R)-2-phenyl-ethyl]-pyridine (CDP-840; Alexander et al., 2002) (57 mg, 0.154 mmol) and the mesylate 3 (181 mg, 0.230 mmol) in anhydrous acetonitrile (2 mL) sodium iodide (23 mg, 0.154 mmol) was added and stirring was continued for 20 hours at room temperature. At this point the LCMS analysis indicated consumption of the starting pyridine-compound. The reaction mixture was filtered and the filtrate was concentrated, the residue redissolved in dichloromethane (10 mL) and washed with deionized water, brine, dried (Na₂SO₄) and concentrated to provide the crude product (211 mg) as yellow oil. Silica-gel chromatography (0-50% gradient CH₂Cl₂/MeOH) afforded the fully protected pyridinium salt (191 mg, 0.179 mmol).

¹H NMR (400 MHz, DMSO-D6) δ ppm 9.03 (m, 1H), 8.79 (m, 1H), 8.00 (m, 2H), 7.23 (dd, J=20.06, 12.55 Hz, 7H), 6.80 (s, 2H), 5.68 (m, 2H), 5.37 (m, 1H), 4.71 (m, 2H), 4.50 (m, 1H), 3.65 (s, 3H), 3.09 (m, 2H), 2.68 (m, 3H), 1.82 (m, 1H), 1.26 (dddd, J=61.30, 60.82, 36.68, 30.44 Hz, 19H); ³¹P NMR (400 MHz, DMSO-d6) δ ppm 67.92 (s, 1P); ES/MS, calcd for C₆₃H₈₈N₂O₁₀P 1063.62 m/z (M)⁺; observed, 1363.7 m/z.

Deprotection step. The purified material from the quaternization step (189 mg, 0.178 mmol) was dissolved in anhydrous dichloromethane (3 mL), which was followed by a dropwise addition of the HCl solution (2 mL, 4N in 1,4-dioxane) with stirring at room temperature After 1 hour the reaction was concentrated, triturated with diethyl ether followed by stirring for 1 hour and filtration. The crude material (143 mg) was purified by the reverse-phase chromatography (gradient H₂O/ACN with 1% AcOH, Teledyne Isco 4.3 gram C-18 column) affording the title mutual prodrug (64 mg, 0.075 mmol).

¹H NMR (400 MHz, DMSO-d6) δ ppm 9.14-9.01 (m, 1H), 8.01-7.82 (m, 1H), 7.45-7.05 (m, 6H), 6.97-6.85 (m, 1H), 6.80 (s, 1H), 5.75-5.59 (m, 1H), 4.83-4.66 (m, 1H), 4.66-4.41 (m, 2H), 3.65 (s, 3H), 1.90 (s, 1H), 1.85-1.73 (m, 1H), 1.73-1.35 (m, 6H), 1.27 (s, 2H); ³¹P NMR (400 MHz, DMSO-D6) δ ppm −3.63 (s, 1P); ES/MS, calcd for C₅₀H₆₄N₂O₈P 851.44 m/z (M)⁺; observed, 851.5 m/z. Anal. Calcd: C, 63.27; H, 7.57; N, 2.73. Found: C, 62.58, H, 7.42; N, 3.18.

### Example 38

### 1-[4-(2-tert-Butylamino-1-hydroxy-ethyl)-2-phosphonooxy-benzyl]-4-[2-(3-cyclopentyloxy-4-methoxy-phenyl)-2-phenyl-ethyl]-pyridinium

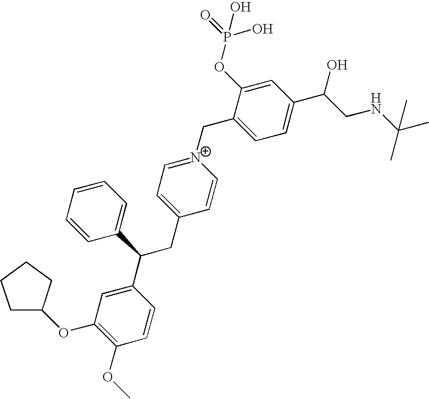

The title compound can be prepared by a two-step procedure described in Example 37, using 4-[2-(3-cyclopentyloxy-4-methoxy-phenyl)-(R)-2-phenyl-ethyl]-pyridine and mesylate 10 as starting materials.

### Example 39

### 4-[2-(3-Cyclopentyloxy-4-methoxy-phenyl)-2-phenyl-ethyl]-1-(4-{1-hydroxy-2-[6-(4-phenyl-butoxy)-hexylamino]-ethyl}-2-phosphonooxymethoxy-benzyl)-pyridinium

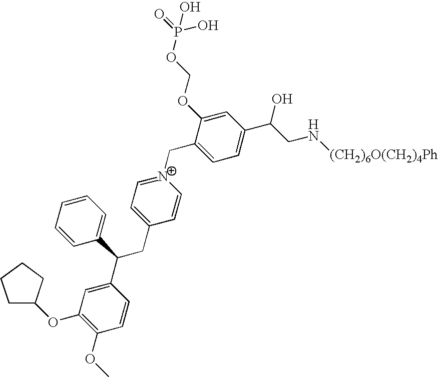

The title compound can be prepared by a two-step procedure described in Example 37, using 4-[2-(3-cyclopentyloxy-4-methoxy-phenyl)-(R)-2-phenyl-ethyl]-pyridine and mesylate 5 as starting materials, except that a TFA/DCM (1:1) mixture is used for a final deprotection carried out at 0° C. for 30 minutes.

### Example 40

### 1-[4-(2-tert-Butylamino-1-hydroxy-ethyl)-2-phosphonooxymethoxy-benzyl]-4-[2-(3-cyclopentyloxy-4-methoxy-phenyl)-2-phenyl-ethyl]-pyridinium

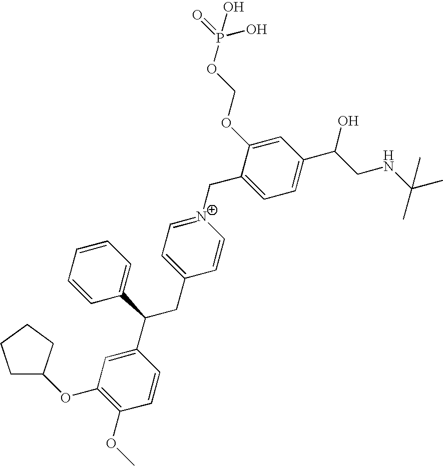

The title compound can be prepared by a two-step procedure described in Example 37, using 4-[2-(3-cyclopentyloxy-4-methoxy-phenyl)-(R)-2-phenyl-ethyl]-pyridine and mesylate 13 as starting materials, except that a TFA/DCM (1:1) mixture is used for a final deprotection carried out at 0° C. for 30 minutes.

### Example 41

### 3,5-Dichloro-4-{2-[1-(4-fluoro-benzyl)-5-hydroxy-1H-indol-3-yl]-2-oxo-acetylamino}-1-(4-{1-hydroxy-2-[6-(4-phenyl-butoxy)-hexylamino]-ethyl}-2-phosphonooxy-benzyl)-pyridinium

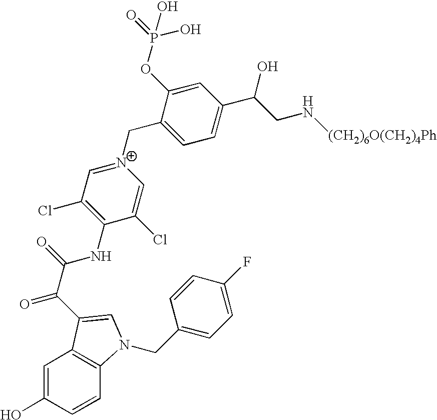

The title compound can be prepared by a two-step procedure described in Example 37, using N-(3,5-dichloro-pyridin-4-yl)-2-[1-(4-fluoro-benzyl)-5-hydroxy-1H-indol-3-yl]-2-oxo-acetamide (AWD 12-281) and mesylate 3 as starting materials.

### Example 42

### 1-[4-(2-tert-Butylamino-1-hydroxy-ethyl)-2-phosphonooxy-benzyl]-3,5-dichloro-4-{2-[1-(4-fluoro-benzyl)-5-hydroxy-1H-indol-3-yl]-2-oxo-acetylamino}-pyridinium

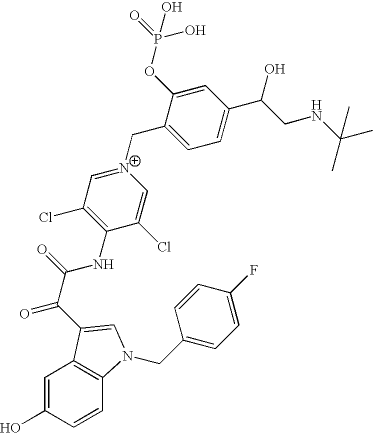

The title compound can be prepared by a two-step procedure described in Example 37, using N-(3,5-dichloro-pyridin-4-yl)-2-[1-(4-fluoro-benzyl)-5-hydroxy-1H-indol-3-yl]-2-oxo-acetamide (AWD 12-281) and mesylate 10 as starting materials.

### Example 43

### 3,5-Dichloro-4-{2-[1-(4-fluoro-benzyl)-5-hydroxy-1H-indol-3-yl]-2-oxo-acetylamino}-1-(4-{1-hydroxy-2-[6-(4-phenyl-butoxy)-hexylamino]-ethyl}-2-phosphonooxymethoxy-benzyl)-pyridinium

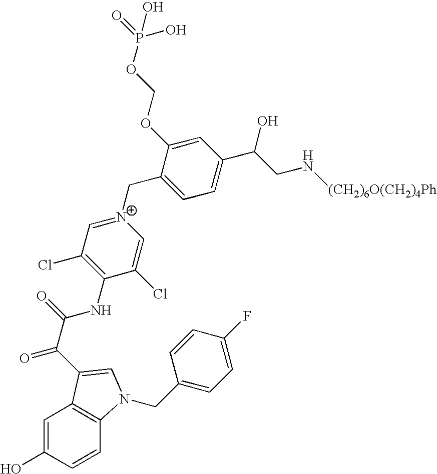

The title compound can be prepared by a two-step procedure described in Example 37, using N-(3,5-dichloro-pyridin-4-yl)-2-[1-(4-fluoro-benzyl)-5-hydroxy-1H-indol-3-yl]-2-oxo-acetamide (AWD 12-281) and mesylate 5 as starting materials, except that a TFA/DCM (1:1) mixture is used for a final deprotection carried out at 0° C. for 30 minutes.

### Example 44

### 1-[4-(2-tert-Butylamino-1-hydroxy-ethyl)-2-phosphonooxymethoxy-benzyl]-3,5-dichloro-4-{2-[1-(4-fluoro-benzyl)-5-hydroxy-1H-indol-3-yl]-2-oxo-acetylamino}-pyridinium

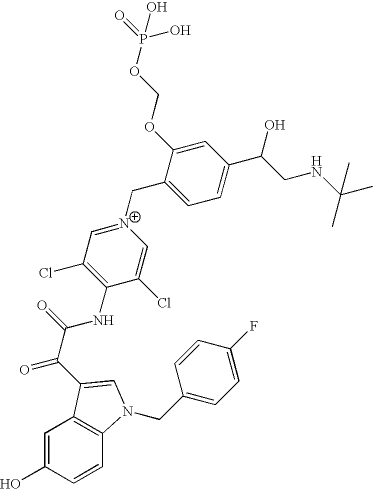

The title compound can be prepared by a two-step procedure described in Example 37, using N-(3,5-dichloro-pyridin-4-yl)-2-[1-(4-fluoro-benzyl)-5-hydroxy-1H-indol-3-yl]-2-oxo-acetamide (AWD 12-281) and mesylate 13 as starting materials, except that a TFA/DCM (1:1) mixture is used for a final deprotection carried out at 0° C. for 30 minutes.

### Example 45

### 5-(3,5-Dichloro-1-oxy-pyridin-4-ylcarbamoyl)-1-(4-{1-hydroxy-2-[6-(4-phenyl-butoxy)-hexylamino]-ethyl}-2-phosphonooxy-benzyl)-8-methoxy-2-trifluoromethyl-quinolinium

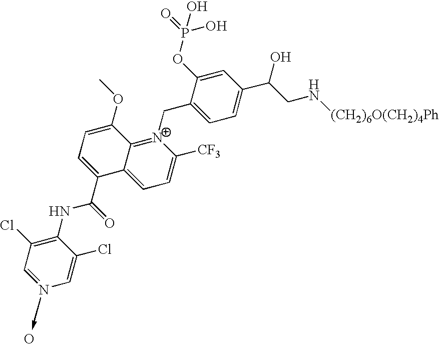

The title compound can be prepared by a two-step procedure described in Example 37, using 8-methoxy-2-trifluoromethyl-quinoline-5-carboxylic acid (3,5-dichloro-1-oxy-pyridin-4-yl)-amide (Sch 351591) and mesylate 3 as starting materials.

### Example 46

### 1-[4-(2-tert-Butylamino-1-hydroxy-ethyl)-2-phosphonooxy-benzyl]-5-(3,5-dichloro-1-oxy-pyridin-4-ylcarbamoyl)-8-methoxy-2-trifluoromethyl-quinolinium

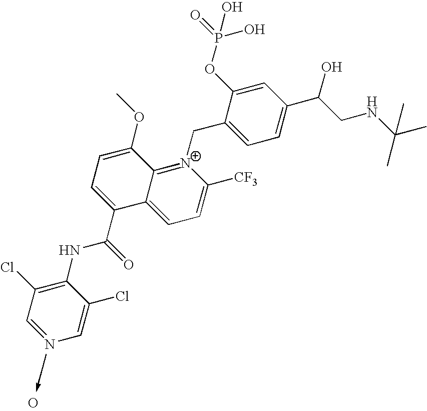

The title compound can be prepared by a two-step procedure described in Example 37, using 8-methoxy-2-trifluoromethyl-quinoline-5-carboxylic acid (3,5-dichloro-1-oxy-pyridin-4-yl)-amide (Sch 351591) and mesylate 10 as starting materials.

### Example 47

### 5-(3,5-Dichloro-1-oxy-pyridin-4-ylcarbamoyl)-1-(4-{1-hydroxy-2-[6-(4-phenyl-butoxy)-hexylamino]-ethyl}-2-phosphonooxymethoxy-benzyl)-8-methoxy-2-trifluoromethyl-quinolinium

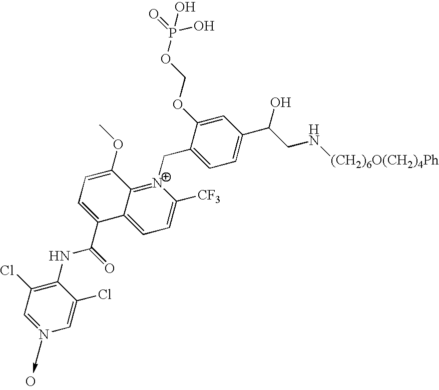

The title compound can be prepared by a two-step procedure described in Example 37, using 8-methoxy-2-trifluoromethyl-quinoline-5-carboxylic acid (3,5-dichloro-1-oxy-pyridin-4-yl)-amide (Sch 351591) and mesylate 5 as starting materials, except that a TFA/DCM (1:1) mixture is used for a final deprotection carried out at 0° C. for 30 minutes.

### Example 48

### 1-[4-(2-tert-Butylamino-1-hydroxy-ethyl)-2-phosphonooxymethoxy-benzyl]-5-(3,5-dichloro-1-oxy-pyridin-4-ylcarbamoyl)-8-methoxy-2-trifluoromethyl-quinolinium

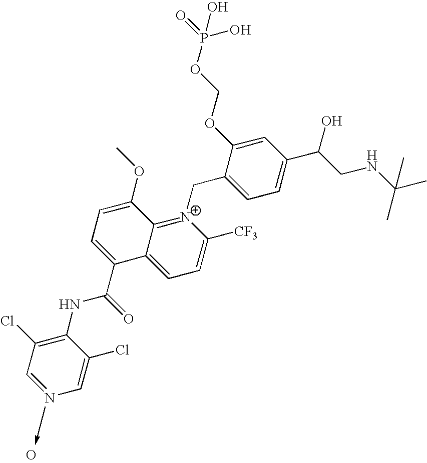

The title compound can be prepared by a two-step procedure described in Example 37, using 8-methoxy-2-trifluoromethyl-quinoline-5-carboxylic acid (3,5-dichloro-1-oxy-pyridin-4-yl)-amide (Sch 351591) and mesylate 5 as starting materials, except that a TFA/DCM (1:1) mixture is used for a final deprotection carried out at 0° C. for 30 minutes.

### Example 49

### 4-[5-(4-Fluoro-phenyl)-2-(4-methanesulfinyl-phenyl)-1H-imidazol-4-yl]-1-(4-{1-hydroxy-2-[6-(4-phenyl-butoxy)-hexylamino]-ethyl}-2-phosphonooxy-benzyl)-pyridinium

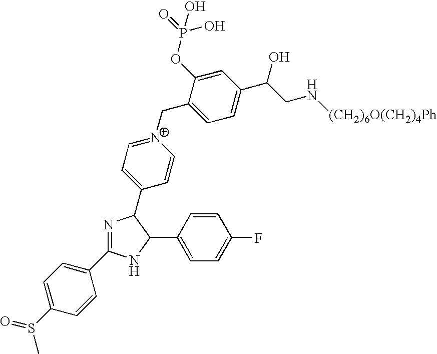

4-[5-(4-Fluoro-phenyl)-2-(4-methanesulfinyl-phenyl)-1H-imidazol-4-yl]-pyridine (SB-203580) can be protected with di-t-butyl-dicarbonate to give the N-imidazole protected 5-(4-fluoro-phenyl)-2-(4-methanesulfinyl-phenyl)-4-pyridin-4-yl-imidazole-1-carboxylic acid tert-butyl ester. That derivative, together with mesylate 3 can be used to synthesize the title mutual prodrug by a two-step procedure described in Example 37.

### Example 50

### 1-[4-(2-tert-Butylamino-1-hydroxy-ethyl)-2-phosphonooxy-benzyl]-4-[5-(4-fluoro-phenyl)-2-(4-methanesulfinyl-phenyl)-1H-imidazol-4-yl]-pyridinium

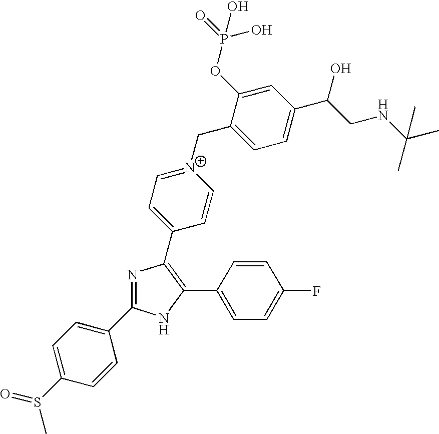

The title compound can be synthesized from 5-(4-fluoro-phenyl)-2-(4-methanesulfinyl-phenyl)-4-pyridin-4-yl-imidazole-1-carboxylic acid tert-butyl ester and mesylate 10, applying the two-step procedure described in Example 37.

### Example 51

### 4-[5-(4-Fluoro-phenyl)-2-(4-methanesulfinyl-phenyl)-1H-imidazol-4-yl]-1-(4-{1-hydroxy-2-[6-(4-phenyl-butoxy)-hexylamino]-ethyl}-2-phosphonooxymethoxy-benzyl)-pyridinium

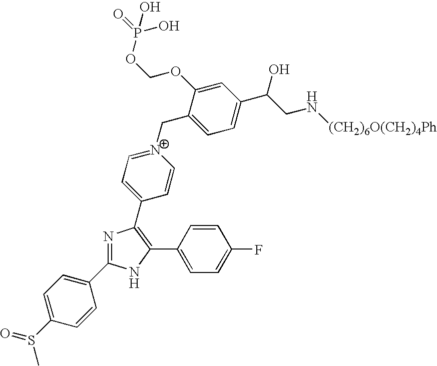

The title compound can be synthesized from 5-(4-fluoro-phenyl)-2-(4-methanesulfinyl-phenyl)-4-pyridin-4-yl-imidazole-1-carboxylic acid tert-butyl ester and mesylate 5, applying the two-step procedure described in Example 37, except that a TFA/DCM (1:1) mixture is used for a final deprotection carried out at 0° C. for 30 minutes.

### Example 52

### 1-[4-(2-tert-Butylamino-1-hydroxy-ethyl)-2-phosphonooxymethoxy-benzyl]-4-[5-(4-fluoro-phenyl)-2-(4-methanesulfinyl-phenyl)-1H-imidazol-4-yl]-pyridinium

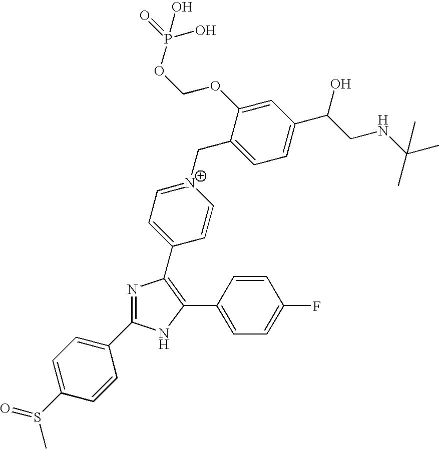

The title compound can be synthesized from 5-(4-fluoro-phenyl)-2-(4-methanesulfinyl-phenyl)-4-pyridin-4-yl-imidazole-1-carboxylic acid tert-butyl ester and mesylate 13, applying the two-step procedure described in Example 37, except that a TFA/DCM (1:1) mixture is used for a final deprotection carried out at 0° C. for 30 minutes.

### Example 53

### 4-[5-(4-Fluoro-phenyl)-2-(4-hydroxy-but-1-ynyl)-3-(3-phenyl-propyl)-3H-imidazol-4-yl]-1-(4-{1-hydroxy-2-[6-(4-phenyl-butoxy)-hexylamino]-ethyl}-2-phosphonooxy-benzyl)-pyridinium

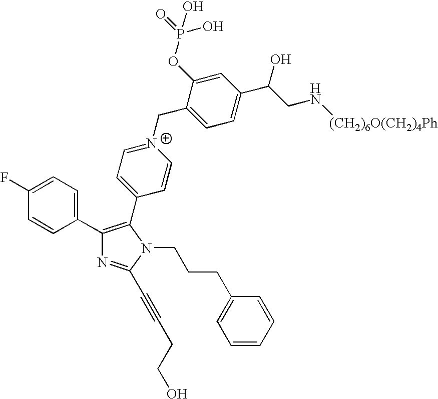

The title compound can be synthesized from 4-[4-(4-fluoro-phenyl)-1-(3-phenyl-propyl)-5-pyridin-4-yl-1H-imidazol-2-yl]-but-3-yn-1-ol (RWJ-67657) and mesylate 3, applying the two-step procedure described in Example 37.

### Example 54

### 1-[4-(2-tert-Butylamino-1-hydroxy-ethyl)-2-phosphonooxy-benzyl]-4-[5-(4-fluoro-phenyl)-2-(4-hydroxy-but-1-ynyl)-3-(3-phenyl-propyl)-3H-imidazol-4-yl]-pyridinium

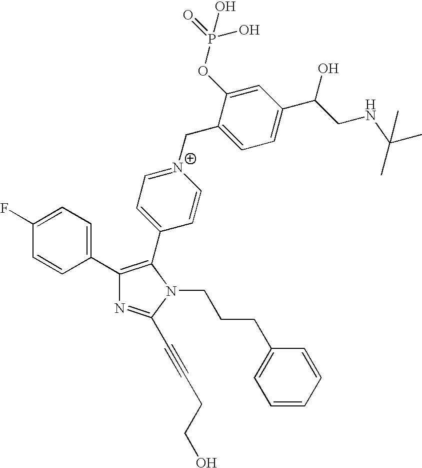

The title compound can be synthesized from 4-[4-(4-fluoro-phenyl)-1-(3-phenyl-propyl)-5-pyridin-4-yl-1H-imidazol-2-yl]-but-3-yn-1-ol (RWJ-67657) and mesylate 10, applying the two-step procedure described in Example 37.

### Example 55

### 4-[5-(4-Fluoro-phenyl)-2-(4-hydroxy-but-1-ynyl)-3-(3-phenyl-propyl)-3H-imidazol-4-yl]-1-(4-{1-hydroxy-2-[6-(4-phenyl-butoxy)-hexylamino]-ethyl}-2-phosphonooxymethoxy-benzyl)-pyridinium

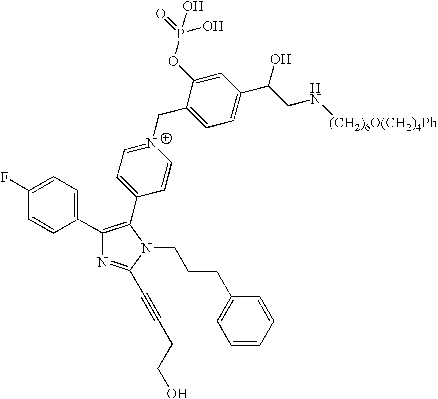

The title compound can be synthesized from 4-[4-(4-fluoro-phenyl)-1-(3-phenyl-propyl)-5-pyridin-4-yl-1H-imidazol-2-yl]-but-3-yn-1-ol (RWJ-67657) and mesylate 5, applying the two-step procedure described in Example 37, except that a TFA/DCM (1:1) mixture is used for a final deprotection carried out at 0° C. for 30 minutes.

### Example 56

### 1-[4-(2-tert-Butylamino-1-hydroxy-ethyl)-2-phosphonooxymethoxy-benzyl]-4-[5-(4-fluoro-phenyl)-2-(4-hydroxy-but-1-ynyl)-3-(3-phenyl-propyl)-3H-imidazol-4-yl]-pyridinium

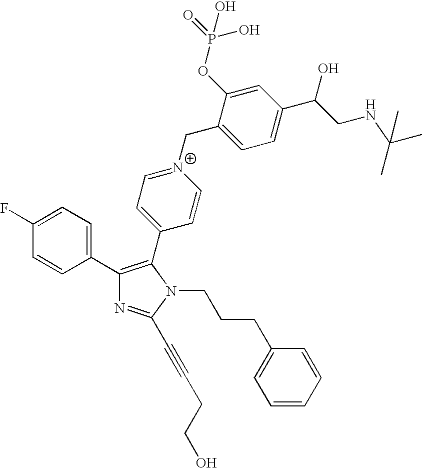

The title compound can be synthesized from 4-[4-(4-fluoro-phenyl)-1-(3-phenyl-propyl)-5-pyridin-4-yl-1H-imidazol-2-yl]-but-3-yn-1-ol (RWJ-67657) and mesylate 13, applying the two-step procedure described in Example 37, except that a TFA/DCM (1:1) mixture is used for a final deprotection carried out at 0° C. for 30 minutes.

### Example 57

### 3,5-Dichloro-4-[(4-difluoromethoxy-8-methanesulfonylamino-dibenzofuran-1-carbonyl)-amino]-1-(4-{1-hydroxy-2-[6-(4-phenyl-butoxy)-hexylamino]-ethyl}-2-phosphonooxy-benzyl)-pyridinium

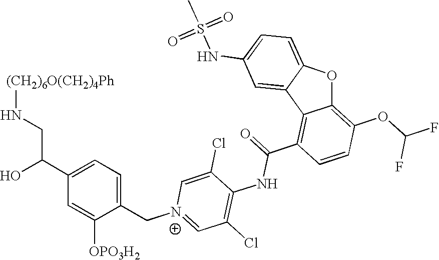

The title compound can be synthesized from 4-difluoromethoxy-8-methanesulfonylamino-dibenzofuran-1-carboxylic acid (3,5-dichloro-pyridin-4-yl)-amide (Oglemilast) and mesylate 3, applying the two-step procedure described in Example 37.

### Example 58

### 1-[4-(2-tert-Butylamino-1-hydroxy-ethyl)-2-phosphonooxy-benzyl]-3,5-dichloro-4-[(4-difluoromethoxy-8-methanesulfonylamino-dibenzofuran-1-carbonyl)-amino]-pyridinium

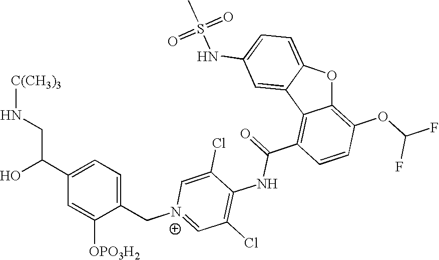

The title compound can be synthesized from 4-difluoromethoxy-8-methanesulfonylamino-dibenzofuran-1-carboxylic acid (3,5-dichloro-pyridin-4-yl)-amide (Oglemilast) and mesylate 10, applying the two-step procedure described in Example 37.

### Example 59

### 3,5-Dichloro-4-[(4-difluoromethoxy-8-methanesulfonylamino-dibenzofuran-1-carbonyl)-amino]-1-(4-{1-hydroxy-2-[6-(4-phenyl-butoxy)-hexylamino]-ethyl}-2-phosphonooxymethoxy-benzyl)-pyridinium

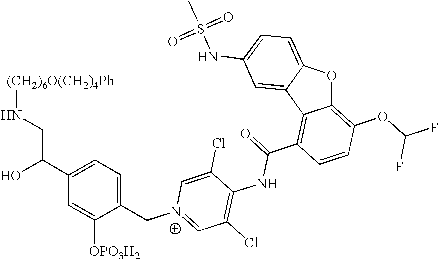

The title compound can be synthesized from 4-difluoromethoxy-8-methanesulfonylamino-dibenzofuran-1-carboxylic acid (3,5-dichloro-pyridin-4-yl)-amide (Oglemilast) and mesylate 5, applying the two-step procedure described in Example 37, except that a TFA/DCM (1:1) mixture is used for a final deprotection carried out at 0° C. for 30 minutes.

### Example 60

### 1-[4-(2-tert-Butylamino-1-hydroxy-ethyl)-2-phosphonooxymethoxy-benz 11]-3,5-dichloro-4-[(4-difluoromethoxy-8-methanesulfonylamino-dibenzofuran-1-carbonyl)-amino]-pyridinium

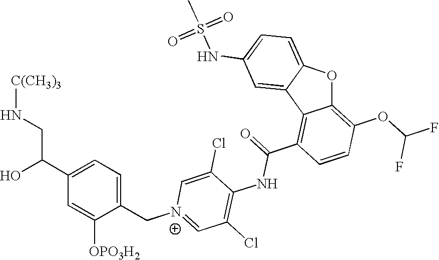

The title compound can be synthesized from 4-difluoromethoxy-8-methanesulfonylamino-dibenzofuran-1-carboxylic acid (3,5-dichloro-pyridin-4-yl)-amide (Oglemilast) and mesylate 13, applying the two-step procedure described in Example 37, except that a TFA/DCM (1:1) mixture is used for a final deprotection carried out at 0° C. for 30 minutes.

### Example 61

### {2-[4-Cyano-4-(3-cyclopentyloxy-4-methoxy-phenyl)-cyclohexanecarbonyloxy]-ethyl}-diethyl-(4-{1-hydroxy-2-[6-(4-phenyl-butoxy)-hexylamino]-ethyl}-2-phosphonooxy-benzyl)-ammonium

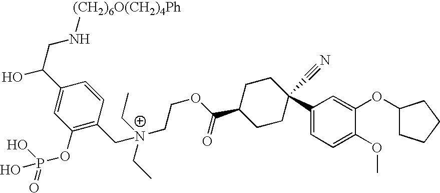

4-Cyano-4-(3-cyclopentyloxy-4-methoxy-phenyl)-cyclohexanecarboxylic acid (Cilomilast) can be esterified with N,N-diethyl-ethanol to yield 4-cyano-4-(3-cyclopentyloxy-4-methoxy-phenyl)-cyclohexanecarboxylic acid 2-diethylamino-ethyl ester.

That ester derivative, together with the mesylate 3, can be used to synthesize the title mutual prodrug applying the two-step procedure described in Example 37.

### Example 62

### [4-(2-tert-Butylamino-1-hydroxy-ethyl)-2-phosphonooxy-benzyl]-{2-[4-cyano-4-(3-cyclopentyloxy-4-methoxy-phenyl)-cyclohexanecarbonyloxy]-ethyl}-diethyl-ammonium

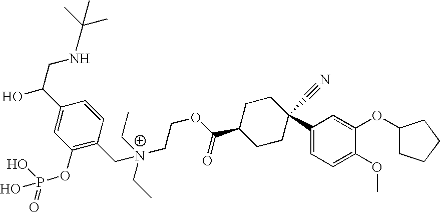

The title compound can be prepared from 4-cyano-4-(3-cyclopentyloxy-4-methoxy-phenyl)-cyclohexanecarboxylic acid 2-diethylamino-ethyl ester and the mesylate 10, applying the two-step procedure described in Example 37.

### Example 63

### {2-[4-Cyano-4-(3-cyclopentyloxy-4-methoxy-phenyl)-cyclohexanecarbonyloxy]-ethyl}-diethyl-(4-{1-hydroxy-2-[6-(4-phenyl-butoxy)-hexylamino]-ethyl}-2-phosphonooxymethoxy-benzyl)-ammonium

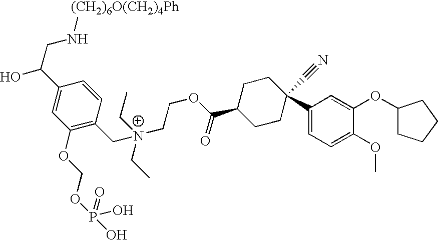

The title compound can be prepared from 4-cyano-4-(3-cyclopentyloxy-4-methoxy-phenyl)-cyclohexanecarboxylic acid 2-diethylamino-ethyl ester and the mesylate 5, applying the two-step procedure described in Example 37, except that a TFA/DCM (1:1) mixture is used for a final deprotection carried out at 0° C. for 30 minutes.

### Example 64

### [4-(2-tert-Butylamino-1-hydroxy-ethyl)-2-phosphonooxymethoxy-benzyl]-{2-[4-cyano-4-(3-cyclopentyloxy-4-methoxy-phenyl)-cyclohexanecarbonyloxy]-ethyl}-diethyl-ammonium

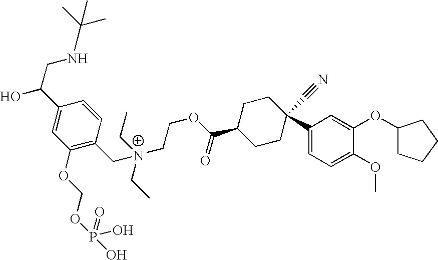

The title compound can be prepared from 4-cyano-4-(3-cyclopentyloxy-4-methoxy-phenyl)-cyclohexanecarboxylic acid 2-diethylamino-ethyl ester and the mesylate 13, applying the two-step procedure described in Example 37, except that a TFA/DCM (1:1) mixture is used for a final deprotection carried out at 0° C. for 30 minutes.

### Example 65

### 4-{3-[4-(3-Chloro-4-fluoro-phenylamino)-7-methoxy-quinazolin-6-yloxy]-propyl}-4-(4-{1-hydroxy-2-[6-(4-phenyl-butoxy)-hexylamino]-ethyl}-2-phosphonooxy-benzyl)-morpholin-4-ium

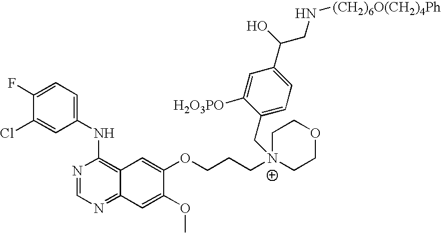

The title compound can be prepared from (3-chloro-4-fluoro-phenyl)-[7-methoxy-6-(3-morpholin-4-yl-propoxy)-quinazolin-4-yl]-amine (Gefitinib) and the mesylate 3, applying the two-step procedure described in Example 37.

### Example 66

### 4-[4-(2-tert-Butylamino-1-hydroxy-ethyl)-2-phosphonooxy-benzyl]-4-{3-[4-(3-chloro-4-fluoro-phenylamino)-7-methoxy-quinazolin-6-yloxy]-propyl}-morpholin-4-ium

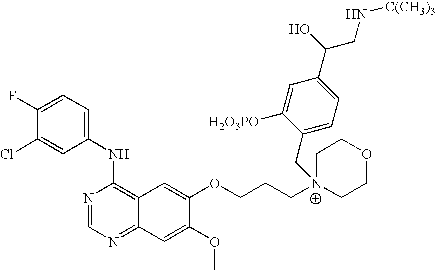

The title compound can be prepared from (3-chloro-4-fluoro-phenyl)-[7-methoxy-6-(3-morpholin-4-yl-propoxy)-quinazolin-4-yl]-amine (Gefitinib) and the mesylate 10, applying the two-step procedure described in Example 37.

### Example 67

### 4-{3-[4-(3-Chloro-4-fluoro-phenylamino)-7-methoxy-quinazolin-6-yloxy]-propyl}-4-(4-{1-hydroxy-2-[6-(4-phenyl-butoxy)-hexylamino]-ethyl}-2-phosphonooxymethoxy-benzyl)-morpholin-4-ium

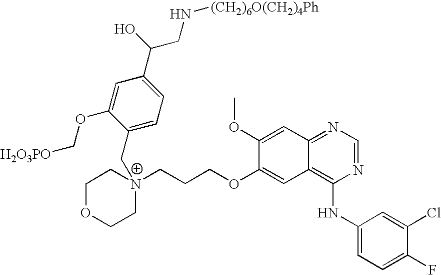

The title compound can be prepared from (3-chloro-4-fluoro-phenyl)-[7-methoxy-6-(3-morpholin-4-yl-propoxy)-quinazolin-4-yl]-amine (Gefitinib) and the mesylate 5, applying the two-step procedure described in Example 37, except that a TFA/DCM (1:1) mixture is used for a final deprotection carried out at 0° C. for 30 minutes.

### Example 68

### 4-[4-(2-tert-Butylamino-1-hydroxy-ethyl)-2-phosphonooxymethoxy-benzyl]-4-{3-[4-(3-chloro-4-fluoro-phenylamino)-7-methoxy-quinazolin-6-yloxy]-propyl}-morpholin-4-ium

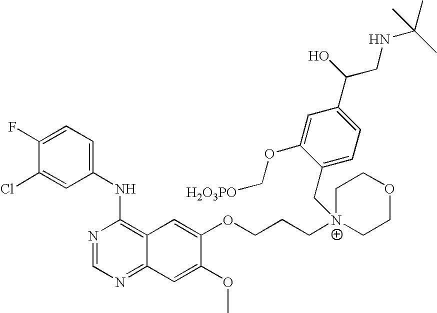

The title compound can be prepared from (3-chloro-4-fluoro-phenyl)-[7-methoxy-6-(3-morpholin-4-yl-propoxy)-quinazolin-4-yl]-amine (Gefitinib) and the mesylate 13, applying the two-step procedure described in Example 37, except that a TFA/DCM (1:1) mixture is used for a final deprotection carried out at 0° C. for 30 minutes.

### Example 69

### 1-(4-{-Hydroxy-2-[6-(4-phenyl-butoxy)-hexylamino]-ethyl}-2-phosphonooxy-benzyl)-1-methyl-4-{4-[4-methyl-3-(4-pyridin-3-yl-pyrimidin-2-ylamino)-phenylcarbamoyl]-benzyl}-piperazin-1-ium

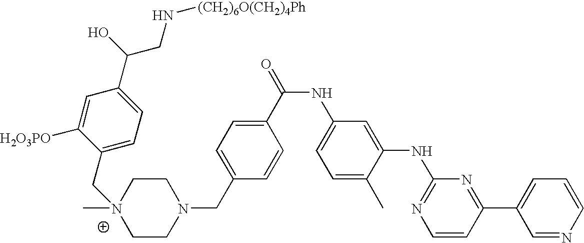

The title compound can be prepared from 4-(4-methyl-piperazin-1-ylmethyl)-N-[4-methyl-3-(4-pyridin-3-yl-pyrimidin-2-ylamino)-phenyl]-benzamide (Imatinib) and the mesylate 3, applying the two-step procedure described in Example 37.

### Example 70

### 1-[4-(2-tert-Butylamino-1-hydroxy-ethyl)-2-phosphonooxy-benzyl]-1-methyl-4-{4-[4-methyl-3-(4-pyridin-3-yl-pyrimidin-2-ylamino)-phenylcarbamoyl]-benzyl}-piperazin-1-ium

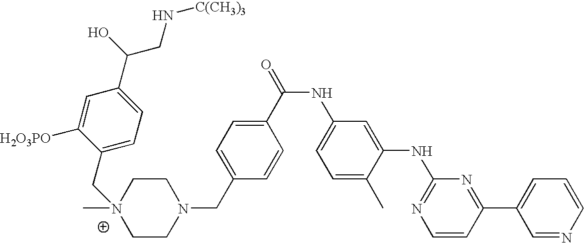

The title compound can be prepared from 4-(4-methyl-piperazin-1-ylmethyl)-N-[4-methyl-3-(4-pyridin-3-yl-pyrimidin-2-ylamino)-phenyl]-benzamide (Imatinib) and the mesylate 10, applying the two-step procedure described in Example 37.

### Example 71

### 1-(4-{1-Hydroxy-2-[6-(4-phenyl-butoxy)-hexylamino]-ethyl}-2-phosphonooxymethoxy-benzyl)-1-methyl-4-{4-[4-methyl-3-(4-pyridin-3-yl-pyrimidin-2-ylamino)-phenylcarbamoyl]-benzyl}-piperazin-1-ium

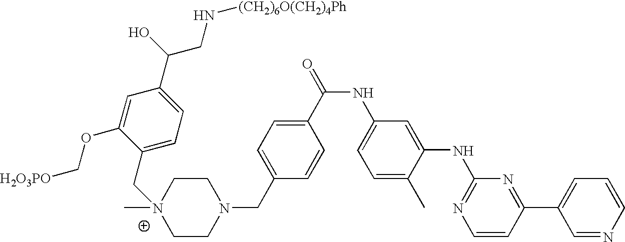

The title compound can be prepared from 4-(4-methyl-piperazin-1-ylmethyl)-N-[4-methyl-3-(4-pyridin-3-yl-pyrimidin-2-ylamino)-phenyl]-benzamide (Imatinib) and the mesylate 5, applying the two-step procedure described in Example 37, except that a TFA/DCM (1:1) mixture is used for a final deprotection carried out at 0° C. for 30 minutes.

### Example 72

### 1-[4-(2-tert-Butylamino-1-hydroxy-ethyl)-2-phosphonooxymethoxy-benzyl]-1-methyl-4-{4-[4-methyl-3-(4-pyridin-3-yl-pyrimidin-2-ylamino)-phenylcarbamoyl]-benzyl}-piperazin-1-ium

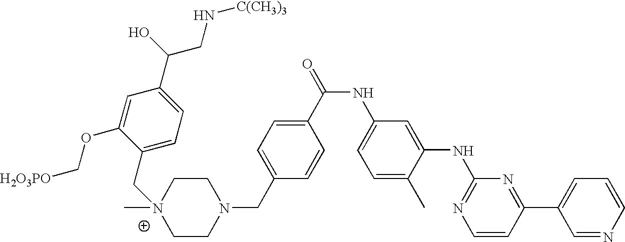

The title compound can be prepared from 4-(4-methyl-piperazin-1-ylmethyl)-N-[4-methyl-3-(4-pyridin-3-yl-pyrimidin-2-ylamino)-phenyl]-benzamide (Imatinib) and the mesylate 13, applying the two-step procedure described in Example 37, except that a TFA/DCM (1:1) mixture is used for a final deprotection carried out at 0° C. for 30 minutes.

### Example 73

### 4-({4-[4-(4-Fluoro-phenylamino)-pyrimidin-2-ylamino]-benzenesulfonyl}-methyl-amino)-1-(4-{1-hydroxy-2-[6-(4-phenyl-butoxy)-hexylamino]-ethyl}-2-phosphonooxy-benzyl)-1-methyl-piperidinium

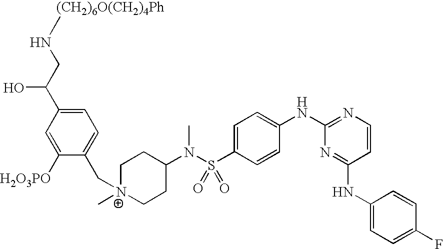

The title compound can be prepared from 4-[4-(4-fluoro-phenylamino)-pyrimidin-2-ylamino]-N-methyl-N-(1-methyl-piperidin-4-yl)-benzenesulfonamide (described by Wagnon et al., 2007) and the mesylate 3, applying the two-step procedure described in Example 37.

### Example 74

### 1-[4-(2-tert-Butylamino-1-hydroxy-ethyl)-2-phosphonooxy-benzyl]-4-({4-[4-(4-fluoro-phenylamino)-pyrimidin-2-ylamino]-benzenesulfonyl}-methyl-amino)-1-methyl-piperidinium

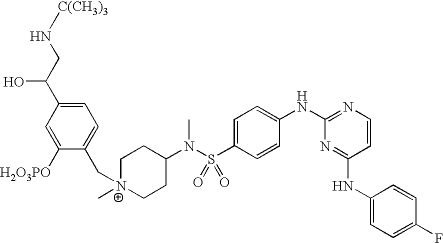

The title compound can be prepared from 4-[4-(4-fluoro-phenylamino)-pyrimidin-2-ylamino]-N-methyl-N-(1-methyl-piperidin-4-yl)-benzenesulfonamide and the mesylate 10, applying the two-step procedure described in Example 37.

### Example 75

### 4-({4-[4-(4-Fluoro-phenylamino-pyrimidin-2-ylamino]-benzenesulfonyl}-methyl-amino)-1-(4-{1-hydroxy-2-[6-(4-phenyl-butoxy)-hexylamino]-ethyl}-2-phosphonooxymethoxy-benzyl)-1-methyl-piperidinium

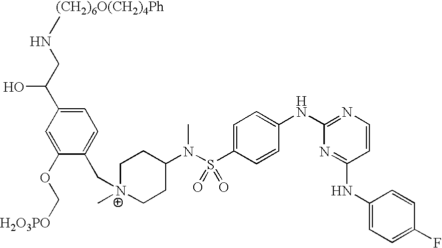

The title compound can be prepared from 4-[4-(4-fluoro-phenylamino)-pyrimidin-2-ylamino]-N-methyl-N-(1-methyl-piperidin-4-yl)-benzenesulfonamide and the mesylate 5, applying the two-step procedure described in Example 37, except that a TFA/DCM (1:1) mixture is used for a final deprotection carried out at 0° C. for 30 minutes.

### Example 76

### 1-[4-(2-tert-Butylamino-1-hydroxy-ethyl)-2-phosphonooxymethoxy-benzyl]-4-({4-[4-(4-fluoro-phenylamino)-pyrimidin-2-ylamino]-benzenesulfonyl}-methyl-amino)-1-methyl-piperidinium

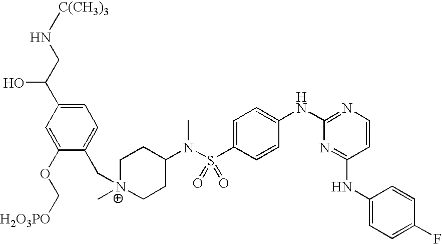

The title compound can be prepared from 4-[4-(4-fluoro-phenylamino)-pyrimidin-2-ylamino]-N-methyl-N-(1-methyl-piperidin-4-yl)-benzenesulfonamide and the mesylate 13, applying the two-step procedure described in Example 37, except that a TFA/DCM (1:1) mixture is used for a final deprotection carried out at 0° C. for 30 minutes.

### Example 77

### 6-Chloro-2-(4-{1-hydroxy-2-[6-(4-phenyl-butoxy)-hexylamino]-ethyl}-2-phosphonooxy-benzyl)-7-methoxy-8-[(2-methyl-pyridine-3-carbonyl)-amino]-9H-b-carbolin-2-ium

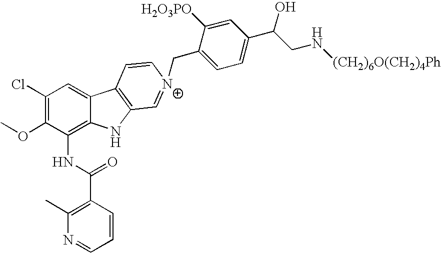

The title compound can be prepared from N-(6-chloro-7-methoxy-9H-b-carbolin-8-yl)-2-methyl-nicotinamide (described by Castro et al., 2003) and the mesylate 3, applying the two-step procedure described in Example 37.

### Example 78

### 2-[4-(2-tert-Butylamino-1-hydroxy-ethyl)-2-phosphonooxy-benzyl]-6-chloro-7-methoxy-8-[(2-methyl-pyridine-3-carbonyl)-amino]-9H-b-carbolin-2-ium

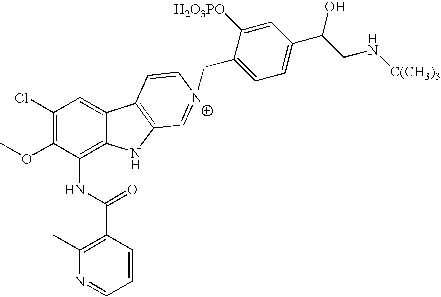

The title compound can be prepared from N-(6-chloro-7-methoxy-9H-b-carbolin-8-yl)-2-methyl-nicotinamide and the mesylate 10, applying the two-step procedure described in Example 37.

### Example 79

### 6-Chloro-2-(4-{1-hydroxy-2-[6-(4-phenyl-butoxy)-hexylamino]-ethyl}-2-phosphonooxymethoxy-benzyl)-7-methoxy-8-[(2-methyl-pyridine-3-carbonyl)-amino]-9H-b-carbolin-2-ium

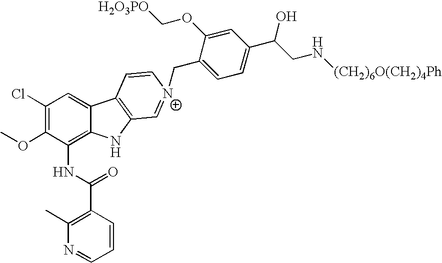

The title compound can be prepared from N-(6-chloro-7-methoxy-9H-b-carbolin-8-yl)-2-methyl-nicotinamide and the mesylate 5, applying the two-step procedure described in Example 37, except that a TFA/DCM (1:1) mixture is used for a final deprotection carried out at 0° C. for 30 minutes.

### Example 80

### 2-[4-(2-tert-Butylamino-1-hydroxy-ethyl)-2-phosphonooxymethoxy-benzyl]-6-chloro-7-methoxy-8-[(2-methyl-pyridine-3-carbonyl)-amino]-9H-b-carbolin-2-ium

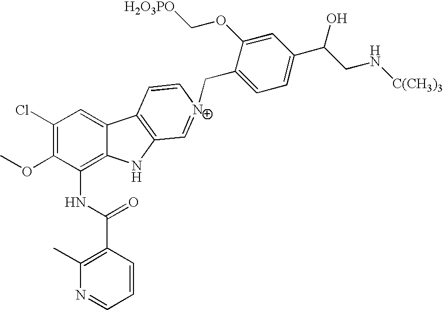

The title compound can be prepared from N-(6-chloro-7-methoxy-9H-b-carbolin-8-yl)-2-methyl-nicotinamide and the mesylate 13, applying the two-step procedure described in Example 37, except that a TFA/DCM (1:1) mixture is used for a final deprotection carried out at 0° C. for 30 minutes.

### Example 81

### Conversion of the Mutual AISTM-β-Agonist Prodrugs (Described in Examples 29 and 37) to Salmeterol and Respective AISTM Drugs after Exposure to Alkaline Phosphatase In Vitro

Preparation of Stock Solutions:

50 mM pH 7.4 tris Buffer Stock Solution

Dissolved 1.500 g (12.5 mmol) tris(hydroxymethyl)aminomethane in ˜200 ml water, added ˜1600 μl of 6M HCl, diluted to 250 ml with water. Final pH=7.45 (measured using a Thermo Orion ROSS pH electrode). Stored at 2°-8° C.

50 mM MgCl₂ Stock Solution

Dissolved 2.033 g (10 mmol) MgCl₂ 6H₂O in 200 ml water to form 50 mM of MgCl₂ solution. Stored at 2°-8° C.

50 mM ZnCl₂ Stock Solution

Dissolved 1.364 g (10 mmol) of ZnCl₂ in 200 ml water. About 0.1 mL of 6 M HCl was added into solution to dissolve insoluble Zn carbonate or hydroxide. Store at 2°-8° C.

Reaction Buffer (pH 7.4, 5 mM tris/1 mM Mg²⁺/1 mM Zn²⁺)

Diluted 5 ml of 50 mM tris stock, 1 ml of 50 mM MgCl₂ stock, and 1 ml of ZnCl₂ and then stocked to 10 ml with water.

Alkaline Phosphatase Stock Solution

Dispersed ˜1 mg (pre-weight) of Sigma P-3895 alkaline phosphatase (Lot number 023K37902) in reaction buffer to make the final concentration of 0.224 mg/mL.

Prodrug Stock Solution

Dissolved ˜2 mg of the mutual prodrug of invention in 10 ml 1:1 acetonitrile/water.

Reaction Product Stock Solution

Dissolved ˜2 mg of each MRA and β-agonist in 20 ml 1:1 acetonitrile/water

Reaction Procedure

The stock solutions were mixed in microcentrifuge tubes, as depicted in the following Table:

| | | Alkaline | Drug | | Reaction | | |
|---|---|---|---|---|---|---|---|
| Solution | Prodrug | phosphatase | standards | | buffer | | 1:1 aq. AcN |
| Blank | — | — | — | | 500 | μl | 500 μl |
| Drug | — | — | 500 | μl | 500 | μl | — |
| standards | | | | | | | |
| Prodrug | 500 μl | — | — | | 500 | μl | — |
| Reaction | 500 μl | 500 μl | 0 | | 0 | | — |

The heat block was set at the 37 degrees. Then 0.5 mL of alkaline phosphatase solution was added into 4 preheated Eppendorf tubes. The aliquot 0.5 of prodrug and drug standards were added into preheated Eppendorf tubes. Immediately after vortexing the aliquots of 25 μL of the all reaction solutions were made into the respective 96-well plate positions. The internal standard (75 μl of 500 ng/mL Glyburide) was added into all samples after each aliquots. That procedure was repeated at every 15 minute intervals for ˜4-5 hours.

The 96-well plates were then analyzed using the LCMS technique.

### HPLC-MS Parameters (Typical)

| LC Gradient | |
|---|---|
| Run time: | 3.0 min |
| Column Flow: | 0.500 ml/mm |
| Gradient | |
| Time (min) | % B |
| 0-0.30 | 15 |
| 1.50 | 95 |
| 2.30 | 95 |
| 2.40 | 15 |
| 3.00 | 15 |
| Mobile Phase A: | 1% formic acid in water |
| Mobile Phase B: | 1% formic acid in acetonitrile |
| Autosampler | |
| Injection Volume: | 5.0 μl |
| Autosample Tray Temperature: | 5 ± 3° C. |
| Column | |
| Phenomenex Synergi Polar RP C₁₈, 4 μm 2.0 × 50 mm | |
| Temperature: | Ambient |

MS DetectorAcquisition Mode
Applied Biosystem API4000 under ESI positive mode

Half Life Calculation (t_{1/2})

In the calculation of half life, we assumed that the disappearance of the mutual prodrug of this invention followed first order kinetics. Therefore,

C=C₀e^{-kt}

ln C=ln C₀−kt

The area peak ratio of prodrug vs IS was plotted against time first; the peak area ratios of later time points were normalized with the peak area ratio of initial time point (ASAP). The natural log of the normalized ratio was then plotted against time to generate a linear curve. The slope of this linear curve k was used for the following calculation.

Graphic plotted rate constant of loss K

At t_{1/2},C₀=2C

t_{1/2}=ln 2/k

### Drug Concentration Determination

Drug concentrations are calculated by normalizing the peak area ration to (t 0). Thus, calculated drug concentrations at any time point=normalized peak area ratio [t (0) mean/t mean]multiplied initial drug concentration. Data (normalized peak area ratio) for the calculations of drug concentrations are listed in Table 1a (ALP activation) and 1b (half-life in ALP and buffer only) for the compound prepared in Example 29, salmeterol, and the ARRY-797 compound (Munson et al., 2004) and Table 2a (ALP activation) and 2b (half-life in ALP and buffer only)) for the compound prepared in Example 37, salmeterol, and the PDE4 inhibitor, CDP-840 (Alexander et al., 2002).

TABLE 1a

| | | | Formation of | | | |
|---|---|---|---|---|---|---|
| | Example 29 | | Sal in ALP | | Formation of | |
| | in ALP (Peak | | (Peak Area | | ARRY-797 | |
| | Area Ratio) | | Ratio) | | in ALP (Peak Area | |
| Time | | Nor- | | Nor- | Ratio) | |
| (mins) | mean | malized | mean | malized | mean | Normalized |
| 0 | 2.3900 | 1.0000 | 0.0471 | 1.0000 | 0.0405 | 1.0000 |
| 15.0 | 2.2200 | 0.9289 | 0.0913 | 1.9405 | 0.0692 | 1.7086 |
| 30.0 | 2.2100 | 0.9247 | 0.1315 | 2.7949 | 0.0928 | 2.2914 |
| 45.0 | 2.0000 | 0.8368 | 0.1565 | 3.3262 | 0.1050 | 2.5926 |
| 60.0 | 1.8050 | 0.7552 | 0.1750 | 3.7194 | 0.1105 | 2.7284 |
| 75.0 | 1.6850 | 0.7050 | 0.1970 | 4.1870 | 0.1185 | 2.9259 |
| 90.0 | 1.5300 | 0.6402 | 0.2245 | 4.7715 | 0.1275 | 3.1481 |
| 105 | 1.4600 | 0.6109 | 0.2420 | 5.1435 | 0.1330 | 3.2840 |
| 120 | 1.5300 | 0.6402 | 0.2805 | 5.9617 | 0.1515 | 3.7407 |
| 135 | 1.4800 | 0.6192 | 0.2870 | 6.0999 | 0.1625 | 4.0123 |
| 150 | 1.4200 | 0.5941 | 0.3285 | 6.9819 | 0.1710 | 4.2222 |
| 165 | 1.4050 | 0.5879 | 0.3395 | 7.2157 | 0.1680 | 4.1481 |
| 180 | 1.3000 | 0.5439 | 0.3525 | 7.4920 | 0.1785 | 4.4074 |
| 210 | 1.2300 | 0.5146 | 0.3970 | 8.4378 | 0.1910 | 4.7160 |
| 240 | 1.1750 | 0.4916 | 0.3870 | 8.2253 | 0.2010 | 4.9630 |
| 270 | 1.2300 | 0.5146 | 0.4575 | 9.7237 | 0.2270 | 5.6049 |

TABLE 1b

| | Added | | | | |
|---|---|---|---|---|---|
| | Compound | | | | |
| | Initial | Calculatcd | | | |
| | Conc. | Compound | | ALP | Half |
| | (μM) in | Final Conc. | Half | Enzyme | Life in |
| | Reaction | (μM) at | Life t_{1/2} | Conc. | Buffer |
| Compound | Mixture | 270 mins. | (mins) | (mg/mL) | Only |
| Example 29 | | | 346.6 | 0.224 | |
| | 111.7 | 57.5 | 141.5 | 0.443 | 3465.7 |
| | | | | | mins |

TABLE 2a

| | | | Formation | | | |
|---|---|---|---|---|---|---|
| | Example 37 | | of Sal in | | Formation of | |
| | in ALP (Peak | | ALP (Peak | | CDP-840 | |
| | Area Ratio) | | Area Ratio) | | in ALP (Peak | |
| Time | | Nor- | | Nor- | Area Ratio) | |
| (mins) | mean | malized | mean | malized | mean | Normalized |
| 0 | 0.8115 | 1.0000 | 0.1205 | 1.0000 | 0.0451 | 1.0000 |
| 15.0 | 0.4160 | 0.5126 | 0.1255 | 1.0415 | 0.1053 | 2.3337 |
| 30.0 | 0.2195 | 0.2705 | 0.1345 | 1.1162 | 0.1710 | 3.7916 |
| 45.0 | 0.1395 | 0.1719 | 0.1360 | 1.1286 | 0.1835 | 4.0687 |
| 60.0 | 0.1039 | 0.1280 | 0.1350 | 1.1203 | 0.2020 | 4.4789 |
| 75.0 | 0.0791 | 0.0974 | 0.1395 | 1.1577 | 0.2250 | 4.9889 |
| 90.0 | 0.0610 | 0.0752 | 0.1465 | 1.2158 | 0.2600 | 5.7650 |
| 105 | 0.0511 | 0.0630 | 0.1475 | 1.2241 | 0.2760 | 6.1197 |
| 120 | 0.0388 | 0.0478 | 0.1470 | 1.2199 | 0.2700 | 5.9867 |
| 135 | 0.0357 | 0.0439 | 0.1505 | 1.2490 | 0.2860 | 6.3415 |
| 150 | 0.0262 | 0.0322 | 0.1465 | 1.2158 | 0.2985 | 6.6186 |
| 165 | 0.0240 | 0.0296 | 0.1645 | 1.3651 | 0.3115 | 6.9069 |
| 180 | 0.0224 | 0.0275 | 0.1805 | 1.4979 | 0.3355 | 7.4390 |
| 210 | 0.0184 | 0.0227 | 0.1785 | 1.4813 | 0.3565 | 7.9047 |
| 240 | 0.0161 | 0.0198 | 0.1810 | 1.5021 | 0.3720 | 8.2483 |
| 270 | 0.0131 | 0.0161 | 0.1830 | 1.5187 | 0.3965 | 8.7916 |

TABLE 2b

| | Added | | | | |
|---|---|---|---|---|---|
| | Compound | Calculated | | ALP | |
| | Initial Conc. | Compound | | Enzyme | Half |
| | (μM) in | Final Conc. | | Conc. | Life in |
| | Reaction | (μM) at | Half Life | (mg/ | Buffer |
| Compound | Mixture | 270 mins. | t_{1/2} (mins) | mL) | Only |
| Example 37 | | | 55.5 | 0.224 | |
| | | | 46.5 | 0.224 | |
| | 117.4 | 1.9 | 24.6 | 0.443 | 385.1 |
| | | | | | mins |

1. A compound of the formula A 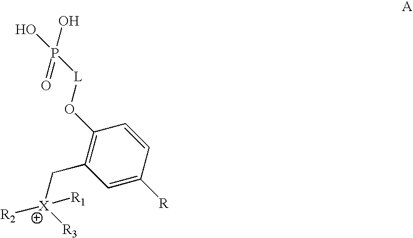
and pharmaceutical acceptable salts thereof, wherein:
X represents a quaternizable moiety;
R₁R₂R₃X taken together represents an anti-inflammatory signal transduction modulator (AISTM) or its prodrug linking the parent molecule possessing AISTM activity to a quaternizable moiety X;
L is a bond or methyleneoxy- (CH₂O) group;
R is 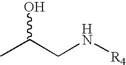
where R₄ is an alkyl group of 1-12 carbon atoms, arylalkyl or substituted arylalkyl with 1-3 CH₂ groups in the carbon chain substituted with atom(s) selected from O, S and NR₅ where R₅ is hydrogen or alkyl.

2. The compound of claim 1 wherein L is a bond.

3. The compound of claim 1 wherein the anti-inflammatory signal transduction modulator is a phosphodiesterase inhibitor.

4. The compound of claim 1 wherein the anti-inflammatory signal transduction modulator is a kinase inhibitor.

5. The compound of claim 1 wherein the anti-inflammatory signal transduction modulator is a transcription factor inhibitor.

6. The compound of claim 1 wherein R₄ is (CH₂)₆O(CH₂)₄Ph or tert-butyl.

7. A compound of claim 1 wherein R₁R₂R₃R₄X is selected from the group consisting of:
5-(2,4-Difluoro-phenoxy)-1-isobutyl-1H-imidazole-6-carboxylic acid-(2-dimethylaminoethyl)-amide;
3-Cyclopropylmethoxy-N-(3,5-dichloropyridin-4-yl)-4-difluoromethoxybenzamide;
4-[2-(3-Cyclopentyloxy-4-methoxyphenyl)-2-phenylethyl]pyridine;
N-(3,5-Dichloro-4-pyridinyl)-4-(difluoromethoxy)-8-[(methylsulfonyl)amino]-1-dibenzofurancarboxamide;
N-(3,5-Dichloropyridin-4-yl)-2-[1-(4-fluorobenzyl)-5-hydroxy-1H-indol-3-yl]-2-oxoacetamide;
8-Methoxy-2-trifluoromethylquinoline-5-carboxylic acid-(3,5-dichloro-1-oxypyridin-4-yl)-amide;
4-[5-(4-Fluorophenyl)-2-(4-methanesulfinylphenyl)-1H-imidazol-4-yl]-pyridine;
4-[4-(4-Fluorophenyl)-1-(3-phenylpropyl)-5-pyridin-4-ul-1H-imidazol-2-yl]-but-3-yn-1-ol;
4-Cyano-4-(3-cyclopentyloxy-4-methoxyphenyl)-cyclohexanecarboxylic acid diethylaminoethyl ester;
(3-Chloro-4-fluorophenyl)-[7-methoxy-6-(3-morpolin-4-yl-propoxy)-quinazolin-4-yl]-amine;
4-(4-Methylpiperazin-1-ylmethyl)-N-[4-methyl-3-(4-pyridin-3-yl-pyrmidin-2-ylamino)-phenyl]-benzamide;
5-{4-[2-(5-Ethylpyridin-2-yl)ethoxy]-benzyl}-thiazolidine-2,4-dione;
5-{4-[2-(5-Methylpyridin-2-ylamino)-ethoxy]-benzyl}-thiazolidine-2,4-dione; and
O-Cyclosporine A-N,Nidiethylglycyl ester.

8. A compound of claim 1 selected from the group consisting of:
(2-{[5-(2,4-Difluorophenoxy)-1-isobutyl-1H-indazole-6-carbonyl]-amino}-ethyl)-(5-{1-hydroxy-2-[6-(4-phenylbutoxy)-hexylamino]-ethyl}-2-phosphonooxybenzyl)-dimethylammonium;
[5-(2-tert-Butylamino-1-hydroxyethyl)-2-phosphonooxybenzyl]-(2-{[5-(2,4-difluorophenoxy)-1-isobutyl-1H-indazole-6-carbonyl]-amino}-ethyl)-dimethylammonium;
4-[2-(3-Cyclopentyloxy-4-methoxyphenyl)-2-phenylethyl]-1-(4-{1-hydroxy-2-[6-(4-phenylbutoxy)-hexylamino]-ethyl}-2-phosphonooxybenzyl)-pyridinium;
[4-(2-tert-Butylamino-1-hydroxyethyl)-2-phosphonooxybenzyl]-4-[2-(3-cyclopentyloxy-4-methoxyphenyl)-2-phenylethyl]-pyridinium;
3,5-Dichloro-4-[(4-difluoromethoxy-8-methanesulfonylaminodibenzofuran-1-carbonyl)-amino]-1-(4-{1-hydroxy-2-[6-(4-phenylbutoxy)-hexylamino]ethyl}-2-phosphonooxybenzyl)-pyridinium; and
1-[4-(2-tert-Butylamino-1-hydroxyethyl)-2-phosphonooxybenzyl]-3,5-dichloro-4-[(4-difluoromethoxy-8-methanesulfonylaminodibenzofuran-1-carbonyl)-amino]-pyridinium.

9. (canceled)

10. (canceled)

11. (canceled)

12. (canceled)

13. (canceled)

14. (canceled)

15. (canceled)

16. (canceled)

17. An aerosol formulation of a compound of claim 1 wherein the mutual prodrug is prepared as a dry powder and the formulation is administered using a dry powder inhaler.

18. An aerosol formulation for the prevention and treatment of pulmonary inflammation or bronchoconstriction, said formulation comprising from about 10 μg to about 1000 μg of at least one mutual prodrug of claim 1 wherein said formulation is adapted to be administered by aerosolization to produce predominantly aerosol particles between 1 and 5μ.

19. An aerosol formulation for the prevention and treatment of pulmonary inflammation or bronchoconstriction, said formulation comprising from about 10 μg to about 1000 μg of at least one mutual prodrug of claims 1 prepared as a dry powder for aerosol delivery in a physiologically compatible and tolerable matrix wherein said formulation is adapted to be administered using a dry powder inhaler able to produce predominantly aerosol particles between 1 and 5μ.

20. A method for the prevention and treatment of pulmonary inflammation or bronchoconstriction, comprising administering to a patient in need of such treatment an effective amount of an aerosol formulation comprising about 10 μg to about 1000 μg of at least one monophosphate mutual prodrug as in claim 1.

21. A method as in claim 20 wherein when the mutual prodrug is delivered to the lung, the phosphate group is cleaved by an endogenous enzyme and the AISTM and the β-agonist are individually released in a simultaneous manner.

22. (canceled)
